SEQUENCE LISTING (35 sequences)

SEQ ID NO: 1 - protein, 11 aa, Artificial
  fragment of humanized porcine NTCP
KGIVISLVLVL

SEQ ID NO: 2 - protein, 346 aa, Sus scrofa
MEALNESAPINFTLPHNFGKRPTDLALSVILVFMLLIIMLSLGCTMEFGRIRAHFRKPKGLAIALVAQYGIMPLTAFALGKLFRLNNVEALAILICGCSPGGNLSNIFALAMKGDMNLSIMMTTCSTFLALGMMPLLLYLYSRGIYDGTLKDKVPYGSIVISLILILIPCTIGIILNTKRPQYVRYVIKGGTILLILCAIAVTVLSVLNVGKSILFVMTPHLVATSSLMPFTGFLLGYLLSALFRLNARCSRTVCMETGCQNVQLCSTILNVTFPPEVIGPLFFFPLLYMLFQLGEGLLFIAIFRCYEKTKLSKDKMKTISAADSTEETIPTALGNGTHKGEECPP

SEQ ID NO: 3 - protein, 346 aa, Artificial
  humanized porcine NTCP
MEALNESAPINFTLPHNFGKRPTDLALSVILVFMLLIIMLSLGCTMEFGRIRAHFRKPKGLAIALVAQYGIMPLTAFALGKLFRLNNVEALAILICGCSPGGNLSNIFALAMKGDMNLSIMMTTCSTFLALGMMPLLLYLYSRGIYDGTLKDKVPYKGIVISLVLVLIPCTIGIILNTKRPQYVRYVIKGGTILLILCAIAVTVLSVLNVGKSILFVMTPHLVATSSLMPFTGFLLGYLLSALFRLNARCSRTVCMETGCQNVQLCSTILNVTFPPEVIGPLFFFPLLYMLFQLGEGLLFIAIFRCYEKTKLSKDKMKTISAADSTEETIPTALGNGTHKGEECPP

SEQ ID NO: 4 - protein, 346 aa, Homo sapiens
MEAHNASAPFNFTLPPNFGKRPTDLALSVILVFMLFFIMLSLGCTMEFSKIKAHLWKPKGLAIALVAQYGIMPLTAFVLGKVFRLKNIEALAILVCGCSPGGNLSNVFSLAMKGDMNLSIVMTTCSTFCALGMMPLLLYIYSRGIYDGDLKDKVPYKGIVISLVLVLIPCTIGIVLKSKRPQYMRYVIKGGMIIILLCSVAVTVLSAINVGKSIMFAMTPLLIATSSLMPFIGFLLGYVLSALFCLNGRCRRTVSMETGCQNVQLCSTILNVAFPPEVIGPLFFFPLLYMIFQLGEGLLLIAIFWCYEKFKTPKDKTKMIYTAATTEETIPGALGNGTYKGEDCSP

SEQ ID NO: 5 - protein, 346 aa, Sus scrofa
MEALNESAPINFTLPHNFGKRPTDLALSVILVFMLLIIMLSLGCTMEFGRIRAHFRKPKGLAIALVAQYGIMPLTAFALGKLFRLNNVEALAILICGCSPGGNLSNIFALAMKGDMNLSIMMTTCSTFLALGMMPLLLYLYSRGIYDGTLKDKVPYGSIVISLILIPIPCTIGIILNTKRPQYVRYVIKGGTILLILCAIAVTVLSVLNVGKSILFVMTPHLVATSSLMPFTGFLLGYLLSALFRLNARCSRTVCMETGCQNVQLCSTILNVTFPPEVIGPLFFFPLLYMLFQLGEGLLFIAIFRCYEKTKLSKDKMKTISAADSTEETIPTALGNGTHKGEECPP

SEQ ID NO: 6 - DNA, 35028 bp, Artificial
  Adenovirus hNTCP
aacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcacaagtttgtacaaaaaagcaggctagcattcctgcagcccgggggatccactagcgtttaaacttaagcttcgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggctcgagtgccaccatggaggcccacaacgcgtctgccccattcaacttcaccctgccacccaactttggcaagcgccccacagacctggcactgagcgtcatcctggtgttcatgttgttcttcatcatgctctcgctgggctgcaccatggagttcagcaagatcaaggctcacttatggaagcctaaagggctggccatcgccctggtggcacagtatggcatcatgcccctcacggcctttgtgctgggcaaggtcttccggctgaagaacattgaggcactggccatcttggtctgtggctgctcacctggagggaacctgtccaatgtcttcagtctggccatgaagggggacatgaacctcagcattgtgatgaccacctgctccaccttctgtgcccttggcatgatgcctctcctcctgtacatctactccagggggatctatgatggggacctgaaggacaaggtgccctataaaggcatcgtgatatcactggtcctggttctcattccttgcaccatagggatcgtcctcaaatccaaacggccacaatacatgcgctatgtcatcaagggagggatgatcatcattctcttgtgcagtgtggccgtcacagttctctctgccatcaatgtggggaagagcatcatgtttgccatgacaccactcttgattgccacctcctccctgatgccttttattggctttctgctgggttatgttctctctgctctcttctgcctcaatggacggtgcagacgcactgtcagcatggagactggatgccaaaatgtccaactctgttccaccatcctcaatgtggcctttccacctgaagtcattggaccacttttcttctttcccctcctctacatgattttccagcttggagaagggcttctcctcattgccatattttggtgctatgagaaattcaagactcccaaggataaaacaaaaatgatctacacagctgccacaactgaagaaacaattccaggagctctgggaaatggcacctacaaaggggaggactgctccccttgcacagcctagcatgaagccccttgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggccgctcgagtctagagggccgccaccgcggtggctggggctagagagctcgctagcacccagctttcttgtacaaagtggtgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcacggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgtgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcccgggagagctcgatatcgcatgcggatttaaattaatt

SEQ ID NO: 7 - DNA, 35029 bp, Artificial
  Adenovirus phNTCP
aattaatttaaatccgcatgcgatatcgagctctcccgggaattcggatccttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcacaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccgtgaagctgtccctgatggtcgtcatctacctgcctggacagcatggcctgcaacgcgggcatcccgatgccgccggaagcgagaagaatcataatggggaaggccatccagcctcgcgtcgcagatccgaattcccgggagagctcgatatcgcatgcggatttaaattaattaacatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaaagcttacatcggggctacgctggttagcatagctccgagtatgcgtgtcataatcagtgtgggttcttttgtcaaggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgattcgaaattttctgcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatctttgagaccgcacagggtcttaataagggtgcagagatcctcaggtccttgacaaggtgagtgaatgcagccttcggtttctaccgagtgctgagttatggtaatgggcttttctcccaccatgaccaccaatttctgacgcttggttggcaacttgtagctaaggcggtgtccggtggtattactgtcgtaggtgactttggcctgctttaccagacaaaagataccccttttgcactggtgcaagttaaccatgtcttggagctcttgattcatgcgctgttgctcggccgctgccctgcgtctttctagcaggcgctgctctgtaataattccgtccatttctagagaaacctgaattagaatagcccgtagagttgcttgaattgttcataaaccccacagtagctgcgcctttggcctaataccctaagggttttctaagctcacctcctgttctggtaaacagagttattgaggtctgtccggaaaaagtctggtttacggtcaggcggtaggtgtggtgcagcggccggtgacgcactcgtacgttcccggcaggtaaggagggtggtgttttttctgatggagtagctgagctcggagaggttctctcgtagactcactccgtctgggttgaaactgttgtaaatcacagagggagagatgttaaaagtaccaggtaaggttcgccttggtttgcttgggcgggtgaagacggtggcgtttacaggatggcgataggagccccagtatattttaatttctgtatttattatactcagcacagagatggcaacaaagatcttgatgtaatccaaggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaaggacggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaagagaattcggtttcggtgggcgcggattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacgacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcatttggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcgcgaccggccgtattgactatggcgcaggcgagcttgtgtggagaaacaaagcctggaaagcgcttgtcataggtgcccaaaaaatatggcccacaaccaagatctttgacaatggctttcagttcctgctcactggagcccatggcggcagctgttgttgatgttgcttgcttctttatgttgtggcgttgccggccgagaagggcgtgcgcaggtacacggtttcgatgacgccgcggtgcggccggtgcacacggaccacgtcaaagacttcaaacaaaacataaagaagggtgggctcgtccatgggatccacctcaaaagtcatgtctagcgcgtgggcggagttggcgtagagaaggttttggcccaggtctgtgagtgcgcccatggacataaagttactggagaatgggatgcgccaaagggtgcgatcgcaaagaaactttttctgggtaatgctgtcaactgcggtcttgcctataagcggataggggaagttagcagggtaggcctgtccttcgcgcatggtgggggcaaggtagccaacaaatccagagttgttgtgttggtgtaggatgcccacctgttggtagtccttgtatttagtatcatccaccacctgacggctcatgggctggaagtttctaaagaaggagtacatgcggtccttgtagctctctgggatatagaagccctggtagccaatgttgtagttagctagcatttgtaccaggaaccagtctttggtcatgttacactgggcaacgttgtaaccctccccgtcaactgagcgcttaatttcaaactcgttgggggtaagcaggcggtcattgccaggccagctgacagaagagtcaaaggtaatggccaccttcttaaaggtgtggttgaggtaaaaggttccatctaggtagggtatagagccagagtaggtgtaataagggtcgtagcccgagcccagtgatggggtttccttagtcttaaggcgcgtgaaggcccagccgcggaaagccgcccagttgcgggaggggatggatatgggcacgttggtagcgttggcgggtatagggtagagcatgttggcggcggagagatagtcgttaaaggactggtcgttggtgtcgtttctaagcatggcctcaagcgtggaggcggtgttgtgggccatggggaagaaggtggcgtaaaggcaaatgctatcaaacttaatgctggctccgtcaacccttaggtcatttcctagggagctctgcagaaccatgttaacatccttcctgaagttccactcgtaggtgtatgagcccggcaggagaaggaggtttttaatggcaaagaacttctgaggcacctggatgtggaagggcacatagcgaccattgcccagcaacattgagcggtagcgcaggccagcattgcggtggtggttaaatgggttgacgttgtccatatagtcaagggaccagcgtgctccaaggttaatgtagcagtccactaacccgggagccaccactcgcttgttcatgtagtcgtaggtgtttgggttatcagaaatttttacgttggaaggactgtactttagcttgtcgggcaaatacagcgctatgttggagtacaggaaatttctccacaggttggcatttagattgatttccatggcaaaattatttccaactcttatttcatttttatctgaaaattctgtagcatctttttcccatccattttcctgacctgttttaggttttaccttggtaagagtctctgtattaatcacacctcccagtggaaagcagtaatttggaagttcatcttcagttccatgattttcaataattctaacatctggatcatagctgtcaacagcctgattccacatagaaaagtacctggttctatcaccaatggaatcaagcaaaagctggtatgaaagctctgtgtttctgtcttgcaaatctacaacagcattcaactgcgatgcttggcccgccagaacacccatattacccgtgctgttgtaatacattagaccaataaaattgtccctaaaagcaatgtaattaggcctgttgggcatagattgttggcccattagttctcgtgagttaccttccttaatagtgggcatgtaagaaatatgagtgtctggggtttctatatctacatcttcactgtacaataccactttaggagtcaagttatcaccattgcctgcggtcgcctcagtagttgagaaaaattgcatttccacttgactttctagctttccattttgttgctttacaagaatgccttgccctccattttcatttgtgggttttgcatatgaaccgtaacatggtttcattggggtagtctttttaaggactctcccagctgcatgattaatttcagtttcgtaccactgagattctcctatttgaggttcaggttgaaatgttttatcggcatatttaggtgtttgaccttcgacacctatttgaataccctcctttgtaatatttataccagaataaggcgcctgcccaaatacgtgagttttttgctgctcagcttgctcgtctacttcgtcttcgttgtcatcgtcctcttcttctaggtttatttcaagagcagtagcagcttcatcccattcgcaaggatttggggcacccttgggagccagggcgttgtaggcagtgccagagtagggcttaaaagtagggcccctgtccagcacgccgcggatgtcaaagtacgtggaagccatgtccagcacacggttatcacccacagctagggtgaaccgcgccttgtacgagtacgcagtatcctcacggtccacagggatgaaccgcagcgtcaaacgctgggaccggtctgtggtcacgtcgtgcgtaggcgccaccgtggggtttctaaacttgttattcaggctgaagtacgtctcggtggcgcgggcaaactgcaccagcccggggctcaggtactccgaggcgtcctggcccgagatgtgcatgtaagaccactgcggcatcatcgaaggggtagccatcttggaaagcgggcgcgcggcggctcagcagctcctctggcggcgacatggacgcatacatgacacacatacgacacgttagctattcagaagcatcgtcggcgcttcagggattgcacccccagacccacgatgctgttcagtgtgctttgccagttgccactggctacgggccgcaacgatcgcggaccgctggcggcgcggcgcagggacgcgcggctaggacgggttacaacaacggcggtcgggcctggcagcacaggtttctgctgggtgtcggcggggggaggcaggtccagcgttacgggtgtgtgctggcccagcactccggtagccatgggcgcgatgggacgggtggtgggcaggccttgctttagtgcctcctcgtacgagggaggctcgtctatttgcgtcaccagagtttcttccctgtcggggcgcggacgcttttcgccacgcccctctggagacactgtctccacggccggtggaggctcctctacgggagggcggggatcaagcttactgttaatcttattttgcactgcctggttggccaggtccaccaccccgctaatgccagaggccaggccatctaccaccttttgttggaaattttgctctttcaacttatccctcagcatctggcctgtgctgctgttccaggccttgctgccatagttcttaacggtggaaccgaaatttttaatgccgctccacagcgagccccagctgaaggcgccaccgctcatattgctggtgccgatatcttgccagtttcccatgaacgggcgcgagccgtgtcgcggggccagagacgcaaagttgatgtcttccattctacaaaatagttacaggaccaagcgagcgtgagagtccagactttttattttgatttttccacatgcaacttgtttttaatcagtgtctctgcgcctgcaaggccacggatgcaattccgggcacggcgccaatcgccgcggcgatcagtggaataaggaggggcaggataccgccgcgcatgcgacggtgcgacgcgcgccgccgccggtggtgcgcacgacgcatgccgcccgtcaggccgtggccggccatgcccctcctacggtgcattcttcctcggaatcccggcaccgggaaacggaggcggcaggtgagggccatatctgcaagaaccacaaagaccggcttttaaacgatgctggggtggtagcgcgctgttggcagcaccagggtcctgcctccttcgcgagccaccctgcgcacggaaatcggggccagcacgggctggcgacggcgacggcggcggcgggttccagtggtggttcggcgtcgggtagttgctcgtcttctggggcggtaggtgtagccacgatagccgggggtaggcgcaatggaaggatgtagggcatattcgggcagtagcgcgctggcggcgccgtacttcctcgaaccgcgcgggcgccggggggctgaaacgcgaaacatccacgggtccgtttgcacctccgtagaggtcttggacgcggccgcagcgaccgcctgcaccgcggcatccgccaccgctgaggcaaccggggacgtttgtgtctccatgccctctgtggcggtggcaatactggtgctactggtagtgggtatctgaacgtccacggtctgcacgcccagtcccggcgccacctgcttgattggccgcacgcggacctcgggctccagcccaggttccacggtcattttttccaagacatcttccagtcgctggcgcttgggtaccatcagctgcacggtgggtgccaagtcaccagactcgcgctttaggccgcgcttttcttcggacggtgcaagcgcgggcagcacctgctgcagtgttacgggctttaggctaggtgttgggttgccctcgtccagcggcaacgccagcatgtccttatgccgctttccgtaggcaaactccccgaggcgctcgttggcctgctcaagcaggtcctcgtcgccgtacacctcatcatacacgcgcttgtaggtgcgggtggagcgctcaccgggcgtaaagactacggtggtgccgggtcgcaaaacacgttttacgcgtcgacctttccactgtacccgtcgcctgggcgcggtagcgtgcagcagttccacctcgtcgtcaagttcatcatcatcatctttctttttctttttgacccgctttagctttcggggcttgtaatcctgctcttccttcttcggggggccatagatctccggcgcgatgacctggagcatctcttctttgattttgcgcttggacatagcttcgttgcgcgccgccgccgctggatacatacaacagtacgagtctaagtagttttttcttgcaatctagttgcgcggggggcgggtgcgcacgggcacgcgcaggccgctaaccgagtcgcgcacccaatacacgttgcccctgcgaccctgagtcatagcactaatggccgcggctgctgcggcggccgctcgtcgcctggacctggggggcacagtgacaatacccgcggccagccttcgagcggcccgcatggccgcccgtcggccggtgcgacgtgcgcggttaagcagggccgccgccgcgcgttgggcggcagtgccgggtcggcggcggtggcgacgtgctacgcgcctccgccgtctcttcattttagcatagcgccgggctccgcgcaccacggtctgaatggccgcgtccactgtggacactggtggcggcgtgggcgtgtagttgcgcgcctcctccaccaccgcgtcgatggcgtcatcgacggtggtgcgcccagtgcggccgcgtttgtgcgcgccccagggcgcgcggtagtgcccgcgcacgcgcactgggtgttggtcggagcgcttcttggccccgccaaacatcttgcttgggaagcgcaggccccagcctgtgttattgctgggcgatataaggatggacatgcttgctcaaaaagtgcggctcgataggacgcgcggcgagactatgcccagggccttgtaaacgtaggggcaggtgcggcgtctggcgtcagtaatggtcactcgctggactcctccgatgctgttgcgcagcggtagcgtcccgtgatctgtgagagcaggaacgttttcactgacggtggtgatggtgggggctggcgggcgcgccaaaatctggttctcgggaaagcgattgaacacgtgggtcagagaggtaaactggcggatgagttgggagtagacggcctggtcgttgtagaagctcttggagtgcacgggcaacagctcggcgcccaccaccggaaagttgctgatctggcgcgtggagcggaaggtcacggggtcttgcatcatgtctggcaacgaccagtagacctgctccgagccgcaggttacgtcaggagtgcaaagcagggtccatgagcggattccggtctgagggtcgccgtagttgtatgcaaggtaccagctgcggtactgggtgaaggtgctgtcattgcttattaggttgtaactgcgtttcttgctgtcctctgtcaggggtttgatcaccggtttcttctgaggcttctcgacctcgggttgcgcagcgggggcggcagcttcggccgctgcttcggcctcagcgcgcttctcctcagcccgtgtggcaaaggtgtcgccgcgaatggcatgatcgttcatgtcctccaccggctgcattgccgcggctgccgcgttggagttctcttccgcgccgctgccactgctgttgctgccgcctgcgccacccccgccctgttcggtgtcatctttcaagctcgcctggtaggcgtccacatccaacagtgcgggaatgttaccaccctccagatcatcgtaggtgatcctaaagccctcctggaagggttgccgcttgcggatgcccaacaagttgctcaggcggctgtgggtgaagtccaccccgcatcctggcagcaaaatgatgtctggatggaaggcttcgtttgtatataccccaggcatgacaagaccagtgacggggtcaaaccccagtctgaagttgcgggtgtcaaactttaccccgatgtcgctttccagaaccccgttctgtctgcccactttcaagtagtgctccacgatcgcgttgttcataaggtctatggtcatggtctcggagtagttgccctcgggcagcgtgaactccacccactcgtatttcagctccacctgattgtccttagtaggcaagcgcgacaccatcacccgcgccttaaacttattggtaaacatgaactcgttcacatttggcatgttggtatgcaggatggttttcaggtcgccgccccagtgcgaccggtcgtcaagattgatggtctgtgtgcttgcctcccccgggctgtagtcattgttttgaatgaccgtggtcagaaagttgctgtggtcgttctggtagttcagggatgccacatccgttgacttgttgtccaccaggtacacacgggtggtgtcgaataggggtgccaactcagagtaacggatgctgtttctccccccggtaggccgcaggtaccgcggaggcacaaacggcgggtccaggggagcatcgaagggagaacccagcgccgccgccactggcgccgcgctcaccacactctcgtaggagggaggaggaccttcctcatacatcgccgcgcgccgcatactaaggggaatacaagaaaaccaacgctcggtgccatggccttggtgagttttttattttgcatcatgcttttttttttttaaaacattctccccagcctggggcgaaggtgcgcaaacgggttgccactccctcccaaatccaggacgctgctgtcgtctgccgagtcatcgtcctcccacaccagaccccgctgacggtcgtgcctttgacgacgggtgggcgggcgcgggcctggcacgtccctgtgctcctgcgcgtacgtcttccatctactcatcttgtccactaggctctctatcccgttgttgggaaatgccggaggcaggtttttttcgcgctgcggctgcagcagcgagttgtttaggtactcctcctcgcccagcaggcgcgggcgggtggtgcgagtgctggtaagagaccctatcaagcttggaaatgggctactagcatctgaccgcggggccgcagcgcctagatcggacaagctgcttggcctgcggaagctttcctttcgcagcgccgcctctgcctgctcgcgctgttgcaactctagcagggtctgcggttgcggggaaaacacgctgtcgtctatgtcgtcccagaggaatccatcgttaccctcgggcacctcgaatcccccggtgtagaaaccagggggcggtagccagtgcgggttcaagatggcattggtgaaatactcggggttcacggcggccgcgcgatgcaagtagtccattaggcggttgataaacggccggtttgaggcatacatgcccggttccatgttgcgcgcggtcatgtccagcgccacgctgggcgttaccccgtcgcgcatcaggttaaggctcacgctctgctgcacgtagcgcaaaatgcgctcctcctcgctgtttaaactgtgcaacgaggggatcttctgccgccggttggtcagcaggtagtttagggttgcctccaggctgcccgtgtcctcctgccccagcgcgcggctgacacttgtaatctcctggaaagtatgctcgtccacatgcgcctgacctatggcctcgcggtacagtgtcagcaagtgacctaggtatgtgtcccgggacacgctgccactgtccgtgaagggcgctattagcagcagcaacaggcgcgagttgggcgtcagcaagctagacacggtcgcgcggtcgcctgtgggagcccgcaccccccacagcccctgcaagtttttgaaagcctggctcaggtttacggtctgcaggccttgtctactggtctggaaaaaatagtctggcccagactggtacacctcactttgcggtgtctcagtcaccattagccgcagtgcgctcacaaagttggtgtagtcctcctgtccccgcggcacgttggcgggctgtgtactcaggaaggcgtttagtgcaaccatggagcccaggttgccctgctgctgcgcgcgctcacgctgcgccacggcctcgcgcacatcccccaccagccggtccaggttggtctgcacgttgccgctgttgtaacgagccacgcgctgaagcagcgcgtcgtagaccaggccggcctcgtcgggccggatggccctgttttcggccagcgcgtttacgatcgccagcaccttctcgtgcgtggggtttgcgcgcgccgggaccaccgcttccagaattgcggagagccggttggcctgcggctgctgccggaacgcgtcaggattgcgcgcagtcagcgacatgatgcggtccatgacctggcgccagtcgtccgtggagttaaggccggacggctggctctgcagcgccgcccgcaccgccgggtccgttgcgtcttgcatcatctgatcagaaacatcaccgcttagtactcgccgtcctctggctcgtactcatcgtcctcgtcatattcctccacgccgccgacgttgccagcgcgcgcgggtgccaccgccagcccaggtccggccccagctgcctccagggcgcgtcggcttggggcccagcgcaggtcagcgcccgcgtcaaagtaggactcggcctctctatcgccgctgcccgtgccagccagggccctttgcaggctgtgcatcagctcgcggtcgctgagctcgcgccgccggctcacgctcacggccttgtggatgcgctcgttgcgataaacgcccaggtcgtcgctcaaggtaagcaccttcagcgccatgcgcatgtagaacccctcgatctttacctccttgtctatgggaacgtaaggggtatggtatatcttgcgggcgtaaaacttgcccaggctaagcatggaatagttgatggcggccaccttgtcagccaggctcaagctgcgctcctgcaccactatgctctgcaggatgtttatcaaatcgagcagccagcggccctcgggctctactatgtttagcagcgcatccctgaatgcctcgttgtccctgctgtgctgcactataaggaacagctgcgccatgagcggcttgctatttgggttttgctccagcgcgcttacaaagtcccacagatgcatcagtcctatagccacctcctcgcgcgccacaagcgtacgcacgtggttgttaaagcttttttgaaagttaatctcctggttcaccgtctgctcgtatgcggttaccaggtcggcggccgccacgtgtgcgcgcgcgggactaatcccggttcgcgcgtcgggctcaaagtcctcctcgcgcagcaaccgctcgcgattcaggccatgccgcagctcgcgccctgcgtggaactttcgatcccgcatctcctcgggctcctctccctcgcggtcgcgaaacaggttctgccgcggcacgtacgcctcacgcgtatcacgcttcagctgcacccttgggtaccgctcaggagagggcgctcctagccgcgccaggccctcgccctcctccaagtccaggtagtgccgggcccggcgccgcgggggttcgtaatcaccatctgctgccgcgtcaaccgcggatgtcgcccctcctgacgcggtaggaggaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacggtctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgaccatccgccgtctcaagaccgcctactttaattacatcatcagcagcacctccgccagaaacaaccccgaccgccgcccgctgccgcccgccacggtgctcagcctaccttgcgactgtgactggttagacgcctttctcgagaggttttccgatccggtcgatgcggactcgctcaggtccctcggcggcggagtacctacacaacaattgttgagatgcatcgttagcgccgtatccctgccgcatggcagccccccgccaacccataaccgggacatgacgggcggcgtcttccaactgcgcccccgcgagaacggccgcgccgtcaccgagaccatgcgccgtcgccgcggggagatgatcgagcgctttgtcgaccgcctcccggtgcgccgtcgtcgccgccgtgtcccccctcccccaccgccgccagaagaagaagaaggggaggcccttatggaagaggagattgaagaagaagaagaggcccctgtagcctttgagcgcgaggtgcgcgacactgtcgccgagctcatccgtcttctggaggaggagttaaccgtgtcggcgcgcaactcccagtttttcaacttcgccgtggacttctacgaggccatggagcgccttgaggccttgggggatatcaacgaatccacgttgcgacgctgggttatgtacttcttcgtggcagaacacaccgccaccaccctcaactacctctttcagcgcctgcgaaactacgccgtcttcgcccggcacgtggagctcaatctcgcgcaggtggtcatgcgcgcccgcgatgccgaagggggcgtggtctacagccgcgtctggaacgagggaggcctcaacgccttctcgcagctcatggcccgcatttccaacgacctcgccgccaccgtggagcgagccggacgcggagatctccaggaggaagagatcgagcagttcatggccgagatcgcctatcaagacaactcaggagacgtgcaggagattttgcgccaggccgccgtcaacgacaccgaaattgattctgtcgaactctctttcaggctcaagctcaccgggcccgtcgtcttcacgcagaggcgccagattcaggagatcaaccgccgcgtcgtcgcgttcgccagcaacctacgcgcgcagcaccagctcctgcccgcgcgcggcgccgacgtgcccctgccccctctcccggcgggtccggagccccccctacctccgggggctcgcccgcgtcaccgcttttagatgcatcatccaaggacacccccgcggcccaccgcccgccgcgcggtaccgtagtcgcgccgcggggatgcggcctcttgcaagccatcgacgccgccaccaaccagcccctggaaattaggtatcacctggatctagcccgcgccctgacccgtctatgcgaggtaaacctgcaggagctcccgcctgacctgacgccgcgggagctccagaccatggacagctcccatctgcgcgatgttgtcatcaagctccgaccgccgcgcgcggacatctggactttgggctcgcgcggcgtggtggtccgatccaccgtaactcccctcgagcagccagacggtcaaggacaagcagccgaagtagaagaccaccagccaaacccgccaggcgaggggctcaaattcccactctgcttccttgtgcgcggtcgtcaggtcaacctcgtgcaggatgtacagcccgtgcaccgctgccagtactgcgcacgtttttacaaaagccagcacgagtgttcggcccgtcgcagggacttctactttcaccacatcaatagccactcctccaattggtggcgggagatccagttcttcccgatcggctcgcatcctcgcaccgagcgtctctttgtcacctacgatgtagagacctatacttggatgggggcctttgggaagcagctcgtgcccttcatgctggtcatgaagttcggcggagatgagcctctagtgactgccgcgcgagacctagccgcgaaccttggatgggaccgctgggaacaagacccgcttaccttctactgcatcaccccagaaaaaatggccataggtcgccagtttaggacctttcgcgaccacctgcaaatgctaatggcccgtgacctgtggagctcattcgtcgcttccaaccctcatcttgcagactgggccctttcagagcacgggctcagctcccctgaagagctcacctacgaggaacttaaaaaattgccttccatcaagggcatcccgcgcttcttggaactttacattgtgggccacaacatcaacggctttgacgagatcgtgctcgccgcccaggtaattaacaaccgttccgaggtgccgggacccttccgcatcacacgcaactttatgcctcgcgcgggaaagatactcttcaacgatgtcaccttcgccctgccaaatccgcgttccaaaaagcgcacggactttttgctctgggagcagggcggatgcgacgacactgacttcaaataccagtacctcaaagtcatggtcagggacacctttgcgctcacccacacctcgctccggaaggccgcgcaggcatacgcgctacccgtagaaaagggatgctgcgcctaccaggccgtcaaccagttctacatgctaggctcttaccgttcggaggccgacgggtttccgatccaagagtactggaaagaccgcgaagagtttgtcctcaaccgcgagctgtggaaaaaaaagggacaggataagtatgacatcatcaaggaaaccctggactactgcgccctagacgtgcaggtcaccgccgagctggtcaacaagctgcgcgactcctacgcctccttcgtgcgtgacgcggtaggtctcacagacgccagcttcaacgtcttccagcgtccaaccatatcatccaactcacatgccatcttcaggcagatagtcttccgagcagagcagcccgcccgtagcaacctcggtcccgacctcctcgctccctcgcacgaactatacgattacgtgcgcgccagcatccgcggtggaagatgctaccctacatatcttggaatactcagagagcccctctacgtttacgacatttgcggcatgtacgcctccgcgctcacccaccccatgccatggggtcccccactcaacccatacgagcgcgcgcttgccgcccgcgcatggcagcaggcgctagacttgcaaggatgcaagatagactacttcgacgcgcgcctgctgcccggggtctttaccgtggacgcagaccccccggacgagacgcagctagaccccctaccgccattctgctcgcgcaagggcggccgcctctgctggaccaacgagcgcctacgcggagaggtagccaccagcgttgaccttgtcaccctgcacaaccgcggttggcgcgtgcacctggtgcccgacgagcgcaccaccgtctttcccgaatggcggtgcgttgcgcgcgaatacgtgcagctaaacatcgcggccaaggagcgcgccgatcgcgacaaaaaccaaaccctgcgctccatcgccaagttgctgtccaacgccctctacgggtcgtttgccaccaagcttgacaacaaaaagattgtcttttctgaccagatggatgcggccaccctcaaaggcatcaccgcgggccaggtgaatatcaaatcctcctcgtttttggaaactgacaatcttagcgcagaagtcatgcccgcttttcagagggagtactcaccccaacagctggccctcgcagacagcgatgcggaagagagtgaggacgaacgcgcccccacccccttttatagccccccttcaggaacacccggtcacgtggcctacacctacaaaccaatcaccttccttgatgccgaagagggcgacatgtgtcttcacaccctggagcgagtggaccccctagtggacaacgaccgctacccctcccacttagcctccttcgtgctggcctggacgcgagcctttgtctcagagtggtccgagtttctatacgaggaggaccgcggaacaccgctcgaggacaggcctctcaagtctgtatacggggacacggacagccttttcgtcaccgagcgtggacaccggctcatggaaaccagaggtaagaaacgcatcaaaaagcatgggggaaacctggtttttgaccccgaacggccagagctcacctggctcgtggaatgcgagaccgtctgcggggcctgcggcgcggatgcctactccccggaatcggtatttctcgcgcccaagctctacgccctcaaaagtctgcactgcccctcgtgcggcgcctcctccaagggcaagctgcgcgccaagggccacgccgcggaggggctggactatgacaccatggtcaaatgctacctggccgacgcgcagggcgaagaccggcagcgcttcagcaccagcaggaccagcctcaagcgcaccctggccagcgcgcagcccggagcgcaccccttcaccgtgacccagactacgctgacgaggaccctgcgcccgtggaaagacatgaccctggcccgtctggacgagcaccgactactgccgtacagcgaaagccgccccaacccgcgaaacgaggagatatgctggatcgagatgccgtagagcaggtgaccgagctgtgggaccgcctggaactgcttggtcaaacgctcaaaagcatgcctacggcggacggtctcaaaccgttgaaaaactttgcttccttgcaagaactgctatcgctgggcggcgagcgccttctggcggatttggtcagggaaaacatgcgagtcagggacatgcttaacgaagtggcccccctgctcagggatgacggcagctgcagctctcttaactaccagttgcacccggtaataggtgtgatttacgggcccaccggctgcggtaagtcgcagctgctcaggaacctgctttcttcccagctgatctcccctaccccggaaaccgttttcttcatcgccccgcaggtagacatgatccccccatctgaactcaaagcgtgggaaatgcaaatctgtgagggtaactacgcccctgggccggatggaaccattataccgcagtctggcaccctccgcccgcgctttgtaaaaatggcctatgacgatctcatcctggaacacaactatgacgttagtgatcccagaaatatcttcgcccaggccgccgcccgtgggcccattgccatcattatggacgaatgcatggaaaatcttggaggtcacaagggcgtctccaagttcttccacgcatttccttctaagctacatgacaaatttcccaagtgcaccggatacactgtgctggtggttctgcacaacatgaatccccggagggatatggctgggaacatagccaacctaaaaatacagtccaagatgcatctcatatccccacgtatgcacccatcccagcttaaccgctttgtaaacacttacaccaagggcctgcccctggcaatcagcttgctactgaaagacatttttaggcaccacgcccagcgctcctgctacgactggatcatctacaacaccaccccgcagcatgaagctctgcagtggtgctacctccaccccagagacgggcttatgcccatgtatctgaacatccagagtcacctttaccacgtcctggaaaaaatacacaggaccctcaacgaccgagaccgctggtcccgggcctaccgcgcgcgcaaaacccctaaataaagacagcaagacacttgcttgatccaaatccaaacagagtctggttttttatttatgttttaaaccgcattgggaggggaggaagccttcagggcagaaacctgctggcgcagatccaacagctgctgagaaacgacattaagttcccgggtcaaagaatccaattgtgccaaaagagccgtcaacttgtcatcgcgggcggatgaacgggaagctgcactgcttgcaagcgggctcaggaaagcaaagtcagtcacaatcccgcgggcggtggctgcagcggctgaagcggcggcggaggctgcagtctccaacggcgttccagacacggtctcgtaggtcaaggtagtagagtttgcgggcaggacggggcgaccatcaatgctggagcccatcacattctgacgcaccccggcccatgggggcatgcgcgttgtcaaatatgagctcacaatgcttccatcaaacgagttggtgctcatggcggcggcggctgctgcaaaacagatacaaaactacataagacccccaccttatatattctttcccacccttaagccacgcccacacatttcagtgatctgtcgaatcgatcaccactttgtacaagaaagctgggtgctagcgagctctctagccccagccaccgcggtggcggccctctagactcgagcggccctcgacaagcttcgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggctcgagaggatggaggccctcaacgagtccgctcccatcaacttcaccctcccgcacaacttcggcaaacggcccacagacctggctctgagcgtcattctggtgttcatgctgctcatcatcatgctctccctgggctgcaccatggagttcggcaggatcagggcgcactttcggaaacctaagggactggccatcgccctggtggcgcagtacggcatcatgcccctcactgcctttgcactgggcaagctcttccgactgaacaatgtcgaggcgctggccatcctgatctgtggctgctcacctggggggaacctctccaacatcttcgctctggccatgaagggggacatgaacctcagcatcatgatgaccacctgctccaccttccttgccctgggcatgatgcccctcctcctatacctttactccaggggcatctatgatgggaccctgaaggacaaggtgccctataaaggcatcgtgatatcactggtcctggttctcattccctgcaccataggcatcatcctcaacactaaacggccacaatatgtgcgctatgtcatcaagggaggaacgatcctcttgattttgtgcgctattgctgtcacagtgctctctgtcctcaacgtgggcaagagcatcttgttcgtcatgacgccacacttggtggccacctcctcccttatgcctttcaccggcttcctgctaggctaccttctttctgctcttttccgcctcaatgctcggtgcagtcgcactgtctgcatggagactggatgccaaaacgttcaactctgctccaccatcctcaacgtgaccttcccccctgaagtcattggaccactcttcttctttcctctcctctacatgcttttccagcttggagaagggcttctcttcattgccatctttcggtgctatgagaaaaccaagctttccaaagataaaatgaaaacgatctccgcagctgattcaacagaagaaaccattcccacagcactgggaaatggcacccacaaaggggaagagtgccccccaacacagcctagtgttgtctagcatgaagccccttgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagaaacgctagtggatcccccgggctgcaggaatgctagcctgcttttttgtacaaacttgtgatcgatgtcgctagcggccgctcgagaattctagaggtaccggatccaagcttcgactgactataataataaaacgccaactttgacccggaacgcggaaaacacctgagaaaaacacctgggcgagtctccacgtaaacggtcaaagtccccgcggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgtt

SEQ ID NO: 8 - DNA, 36472 bp, Artificial
  Adenovirus hNTCP-tdTomato
aacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcacaagtttgtacaaaaaagcaggctagcattcctgcagcccgggggatccactagcgtttctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgcggccgcttacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacggccatgttgttgtcctcggaggaggcggtgccggagctgccgctgccggtgctgccggtgccatgccccaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcacggagcctccgcctccggatcctccgcctccggctgtgcaaggggagcagtcctcccctttgtaggtgccatttcccagagctcctggaattgtttcttcagttgtggcagctgtgtagatcatttttgttttatccttgggagtcttgaatttctcatagcaccaaaatatggcaatgaggagaagcccttctccaagctggaaaatcatgtagaggaggggaaagaagaaaagtggtccaatgacttcaggtggaaaggccacattgaggatggtggaacagagttggacattttggcatccagtctccatgctgacagtgcgtctgcaccgtccattgaggcagaagagagcagagagaacataacccagcagaaagccaataaaaggcatcagggaggaggtggcaatcaagagtggtgtcatggcaaacatgatgctcttccccacattgatggcagagagaactgtgacggccacactgcacaagagaatgatgatcatccctcccttgatgacatagcgcatgtattgtggccgtttggatttgaggacgatccctatggtgcaaggaatgagaaccaggaccagtgatatcacgatgcctttatagggcaccttgtccttcaggtccccatcatagatccccctggagtagatgtacaggaggagaggcatcatgccaagggcacagaaggtggagcaggtggtcatcacaatgctgaggttcatgtcccccttcatggccagactgaagacattggacaggttccctccaggtgagcagccacagaccaagatggccagtgcctcaatgttcttcagccggaagaccttgcccagcacaaaggccgtgaggggcatgatgccatactgtgccaccagggcgatggccagccctttaggcttccataagtgagccttgatcttgctgaactccatggtgcagcccagcgagagcatgatgaagaacaacatgaacaccaggatgacgctcagtgccaggtctgtggggcgcttgccaaagttgggtggcagggtgaagttgaatggggcagacgcgttgtgggcctccatggtggcactcgagcctgtcaggagcttgtggatctgtgtgacggcttctcctggtgaaggggcttttataccccctccttccaacccaggctgctgatccctgccaagctgactccaaacctgctgattctgattattgacttagtcaacaaaaggagaataagtaacctacacaaatatgaaccttgcctagggagattagagtatcggaacactcgctctacgaaatgtgcagacagacggggatcctctagagctacctgctgatcgcccggcccctgttcaaacatgtcctaatactctgtctctgcaagggtcatcagtagttttccatcttactcaacatcctcccagtgccctcgacctgcagcccgaagcttgtcgagggccgctcgagtctagagggccgccaccgcggtggctggggctagagagctcgctagcacccagctttcttgtacaaagtggtgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcacggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgtgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcccgggagagctcgatatcgcatgcggatttaaattaatt

SEQ ID NO: 9 - DNA, 23 bp, Artificial
  HBV1844F Primre
gttgcccgtttgtcctctaattc

SEQ ID NO: 10 - DNA, 14 bp, Artificial
  HBV1745R Primer
ggagggatacatag

SEQ ID NO: 11 - protein, 10 aa, Artificial
  fragment of humanized pNTCP
GIVISLVLVL

SEQ ID NO: 12 - protein, 346 aa, Artificial
  porcine NTCP humanized at positions 158-167
MEALNESAPINFTLPHNFGKRPTDLALSVILVFMLLIIMLSLGCTMEFGRIRAHFRKPKGLAIALVAQYGIMPLTAFALGKLFRLNNVEALAILICGCSPGGNLSNIFALAMKGDMNLSIMMTTCSTFLALGMMPLLLYLYSRGIYDGTLKDKVPYSGIVISLVLVLIPCTIGIILNTKRPQYVRYVIKGGTILLILCAIAVTVLSVLNVGKSILFVMTPHLVATSSLMPFTGFLLGYLLSALFRLNARCSRTVCMETGCQNVQLCSTILNVTFPPEVIGPLFFFPLLYMLFQLGEGLLFIAIFRCYEKTKLSKDKMKTISAADSTEETIPTALGNGTHKGEECPP

SEQ ID NO: 13 - DNA, 41 bp, Artificial
  result of sequencing
gccctataaaggcattgtgatatcactggtcctggttctca

SEQ ID NO: 14 - DNA, 41 bp, Artificial; identical to SEQ ID NO: 13
  result of sequencing

SEQ ID NO: 15 - protein, 15 aa, Artificial
  fragment of human NTCP
PYKGIVISLVLVLIP

SEQ ID NO: 16 - DNA, 45 bp, Artificial
  fragment of human NTCP
ccctataaaggcatcgtgatatcactggtcctggttctcattcct

SEQ ID NO: 17 - DNA, 45 bp, Artificial
  fragment of humanised pig sequence
ccctatggcggcattgtgatatcactggtcctggttctcattccc

SEQ ID NO: 18 - DNA, 45 bp, Artificial
  fragment of porcine NTCP
ccctatggcagcattgtgatatcactgatcctgattcccattccc

SEQ ID NO: 19 - protein, 15 aa, Artificial
  fragment of porcine NTCP
PYGSIVISLILIPIP

SEQ ID NO: 20 - DNA, 18 bp, Artificial
  NTCP G1
tccaggggcatctatgat

SEQ ID NO: 21 - DNA, 18 bp, Artificial
  NTCP G2
tctatgatgggaccctga

SEQ ID NO: 22 - DNA, 18 bp, Artificial
  NTCP G3
gaaggacaaggtgcccta

SEQ ID NO: 23 - DNA, 18 bp, Artificial
  NTCP G4
ctcctatacctttactcc

SEQ ID NO: 24 - DNA, 18 bp, Artificial
  NTCP G5
atcatcctcaacactaaa

SEQ ID NO: 25 - DNA, 21 bp, Artificial
  left primer
aagcccttgtcagttgcatca

SEQ ID NO: 26 - DNA, 25 bp, Artificial
  right primer
tcctccactgtataggtgaaaccaa

SEQ ID NO: 27 - DNA, 20 bp, Artificial
  left primer
ctgggctttccacatgcttc

SEQ ID NO: 28 - DNA, 20 bp, Artificial
  right primer
gtgctgggaggacatgatgc

SEQ ID NO: 29 - DNA, 22 bp, Artificial
  left primer
tgcttcactttgcactctcgtg

SEQ ID NO: 30 - DNA, 20 bp, Artificial
  right primer
gctatgtggaagcccaaggc

SEQ ID NO: 31 - DNA, 20 bp, Artificial
  left primer
agggaggcccaaggagaaag

SEQ ID NO: 32 - DNA, 20 bp, Artificial
  right primer
ggtgaggttagtgggggcaa

SEQ ID NO: 33 - DNA, 26 bp, Artificial
  left primer
tggagaaatagcacctacagacttgc

SEQ ID NO: 34 - DNA, 23 bp, Artificial
  right primer
taaggcatgtcatttgggttttt

SEQ ID NO: 35 - DNA, 2900 bp, Artificial
  hNTCP oligo
ttttgctgttgagttgtataagttgcttatatattctagagattaagcccttgtcagttgcatcatttgaaactattttctcccattctgtaagttgtctagtaaaccatattttaaacagatgtgccatcacccaggctttgttgctccatttatatagcacagagtagatacagcataattcttaggggccctaggatttttggaataagcatgggcttcagcttaaagtcaccaggtgcgttagcccctaaggagagagtcagcctgtcctttgaagatttgaagccaagcactgacttttctctagctacgaaaggcctggatggcatcttcttccaatcttctttatccacattgagaatctgttgtttagtgtggccaccttcactgatgatcttagctaggtcttctggataacttgctgcttcactttgcactctcgtgatacggagataacttctttccttaagcctcatgaatcaacctctgctagcttcaaacttttcttccgcagcttcctcatctctctcagccttcacagaattcaagagttagggccttggtctggattaggctttggcttaagggagtgtcatgtctggtttgatctttccagaccattgaaattttctccacatcagcattaaggctgctttgctttcttatcatacttgtgttcactagagtagcacttttaatgtccttcaagaacttctcctttgcatttatgacttggctaactggtgcaagagtcctagctttcagcctatctgggctttccacatgcttccctcactgagattaatcatttttagctttgtttgccacacccgcagcatatggaagtttctgggccaggaatcaaacccacatcacagcagcacctgaaccactacagtgacaatgccagattcttaacccactaagccacaagaggactccaatcatttctagattttgacttgaagtaatagacatgcaattcttcctttcacttgaacacttacaggccattgtgggttagtaattggcctactttcaatattattgtgtctcaggagtagggaggcccaaggagaaagagaagggggaacagtcagtgagtggaatagtcagaacacacacatttatcctttatgttcactgccttaacacagatgagattcacagtgcccccaaaacaattacaacagtaatatcaaaaaatcactctaatagtgtaagagcctgaaatacggtgagcctcaccaaaatgtgagagacatgaagtaagcaaatacatttggagaaatagcacctacagacttgcttaatgcatagctgctaaaacttcactttgttaaaaaccaaacaaacgaaacaccatctgcaaagcataatcaaacgaggtgtgcctgtactaatgagctctctctctacactcttcctgtctcctgcccagcatcatgatgaccacctgctccaccttccttgccctgggcatgatgcccctcctcctatacctttactccaggggcatctatgatgggaccctgaaggacaaggtgccctataaaggcattgtgatatcactggtcctggttctcattccctgcaccataggcatcatcctcaacactaaacggccacaatatgtgcgctatgtcatcaaggtaagaacctgggggcctgggcagggagcaaaaacatggccatgaatggtataaggcatgtcatttgggttttttctgtgtcagttttccttccttgtttaaaaaaaaaaaaaaagtctgctattcgtgggtttgtaatgtacatgagagtagctttgaagcattaggaggaaaaaaagtaaaacactgcacacaactataatgtataatcattttaatactaaaattaaacttttatcatttaactactagatgttaagttttatgctacactctctgtggattcactatcctcaaagagtttatttgcagaacatcaaataacacaggagaaacaagctccaacacagtgtaccatcaagtattggcagaacccaacggggtgaggttagtgggggcaaggtggtcaagcaagccttccttgggggatgggaggaggggctggacttgagctggattatgaaaaaaaaggtagatttgggtaagcctagggagatgcctgaacagaggggttcacaggcagaccaagggcaaactcgactgactggatgtgacactggaagggacaagcccaggggaagaggcagtaggcacacttggaagtatttcagaagtggggtgggaggagggtggagtgagctggcccagaggacatgggggagtgctgggaggacatgatgccactgccttgcctttggaaaaaaatcacattcccagagtgaaccccctgccaattcttgtgaccttagagcccagtccccatcagtctcccctctggcccctggggtctaaggctccaccctggtgctcagaaaaaactctgcctcctaatctcctgagagcctgttttcgtccttgtccctggagccatgcctttgaggcaggctatagagcctgctatgtggaagcccaaggctttgggcctgggacaaagcagctgcaagtcaggttcccaaggtcttttgattttcacgctactgttgtcaagggtagtttctgctcttgccctggcatagtcctaatatcatgatcacaaaaaaccggaagtgtgtttgggtccaccatttcacttacgcaagatactttgtagtgtacaggcataaaggaaaaaaacacaaaacataaaaatgtgaagtgacccatgatatcactaatgtaattttgtcctcctccactgtataggtgaaaccaaagactaaaactccaagtgtaattgatgtaagaacatgtcttcttacctcagaa

(19) United States
(12) Patent Application Publication
(10) Pub. No.: US 2021/0269499 A1
(43) Pub. Date: Sep. 2, 2021

(54) HEPATITIS B AND/OR HEPATITIS D-PERMISSIVE CELLS AND ANIMALS

(71) Applicant: TECHNISCHE UNIVERSITAET MUENCHEN, Muenchen (DE)

(72) Inventors: Ulrike PROTZER, Muenchen (DE); Jochen WETTENGEL, Wertheim (DE); Samuel JESKE, Erding (DE); Konrad FISCHER, Zahling (DE); Angelika SCHNIEKE, Murnau (DE)

(73) Assignee: TECHNISCHE UNIVERSITAET MUENCHEN, Muenchen (DE)

(21) Appl. No.: 17/254,762

(22) PCT Filed: Jun. 21, 2019
(86) PCT No.: PCT/EP2019/066449

§ 371 Date: Dec. 21, 2020

(30) Foreign Application Priority Data
Jun. 21, 2018 (EP) 18178982.7

(51) Int. Cl. C07K 14/705; C12N 15/90

(52) U.S. Cl. CPC C07K 14/705; C12N 2310/20; C12N 15/907

(57) ABSTRACT

The present invention relates to a porcine sodium taurocholate cotransporter polypeptide (NTCP) mutein, which has been modified at sequence positions 157-167 with the human sequence. This NTCP mutein renders a host cell and a transgenic animal susceptible for an infection with hepatitis B virus (HBV) and/or hepatitis D virus (HDV). The present invention further relates to a nucleic acid and a vector comprising the NTCP mutein of the invention. Also presented are methods for producing cells and transgenic animals, which are susceptible to HBV and/or HDV as well as uses of the NTCP mutein screening for compounds or rendering a cell susceptible for an infection with HBV and/or HDV. Additionally provided is a method for identifying a compound, which is useful in the prevention and/or treatment of HBV and/or HDV infection.

### CROSS-REFERENCE TO RELATED APPLICATIONS

The present application claims the benefit of priority of European Patent Application No. 18178982.7 filed 21 Jun. 2018, the content of which is hereby incorporated by reference it its entirety for all purposes.

### TECHNICAL FIELD OF THE INVENTION

The present invention relates to a porcine sodium taurocholate cotransporter polypeptide (NTCP) mutein, which has been modified at sequence positions 157-167 with the human sequence. This NTCP mutein renders a host cell and a transgenic animal susceptible for an infection with hepatitis B virus (HBV) and/or hepatitis D virus (HDV). The present invention further relates to a nucleic acid and a vector comprising the NTCP mutein of the invention. Also presented are methods for producing cells or transgenic animals, which are susceptible to an HBV and/or HDV as well as uses of the NTCP mutein screening for compounds or rendering a cell susceptible for an infection with HBV and/or HDV. Additionally provided is a method for identifying a compound, which is useful in the prevention and/or treatment of HBV and/or HDV infection.

### BACKGROUND

Hepatitis B virus (hereinafter referred to as HBV) is a liver-specific virus that causes hepatitis B in an infected person. More than 240 million people worldwide are infected with HBV (Xia et al., 2017). According to current estimates, more than 680,000 people die each year from hepatitis B or the consequences thereof (Li et al., 2017). Despite an existing vaccination against HBV, these numbers illustrate the need for a new therapy. Although cell culture models can be considered for the selection of pharmacologically relevant substances and inhibitory mechanisms, they can only be used to a very limited extent for subsequent drug development. Pharmaceutical research is therefore largely dependent on the use of animal models in order to imitate the complex processes of infection and subsequent therapy (Dandri et al., 2017).

Due to its evolutionary adaptation to humans, HBV can only infect close relatives such as chimpanzees or, under certain experimental conditions, tree shrews (Tupaia belangeri) in vivo (Mason et al., 2015). Since research on chimpanzees is subject to extremely high ethical and cost restrictions, current approaches are aimed at establishing a different animal model for HBV infection.

Recent research has shown that the species barrier of HBV is mainly based on species-specific changes in the amino acid sequence and thus the 3D structure of the HBV surface receptor (Yan et al., 2012). This surface receptor, actually a bile acid transporter and known as Na⁺ taurocholate co-transporting polypeptide (hereafter NTCP), is specifically expressed on liver cells, binds HBV to the cell surface and leads to internalization and infection of the cells (Yan et al., 2012). It could be shown that the expression of a human NTCP receptor (hereinafter referred to as hNTCP) on liver cells of monkeys or pigs, for example, makes these cells permissive for HBV and thus enables infection of non-human primary cells (Lempp et al., 2017). This lays the foundation for the establishment and generation of HBV-permissive animal models. However, since additional expression of hNTCP, presumably due to increased bile acid transport in the cells, causes problems within the modified animals, the best way to generate an HBV animal model is to modify the non-human NTCP receptor so that HBV can bind to such a chimeric receptor and the actual bile acid receptor function is not impaired or enhanced.

So far, research has been carried out in mice and the so-called Old Word monkeys (Cercopithecidae) to find out which of the evolutionary changes in the NTCP gene blocks the binding of HBV. The aim of these efforts was to humanize NTCP in transgenic animals and thus enable infection with HBV. In mice, for example, mutations in amino acids H84R, T86K, and S87N are essential for HBV binding (He et al., 2016). Despite the generation of a transgenic mouse with this modified NTCP, it is not yet possible to infect a mouse with HBV because other factors are missing intracellularly or block an infection. Therefore, the research cannot fall back on an HBV mouse animal model. Similarly, it could be shown in the Old World monkeys that changes in amino acids G157K, R158G, I160V, L161I and P165L (hereinafter referred to as 157-165) are responsible for HBV binding and that a chimeric monkey NTCP receptor (hereinafter referred to as mcNTCP) with the humanized amino acids 157-165 enables HBV infection (Watashi et al, 2014 and Yan et al., 2013).

As outlined above, mice and Old Word monkeys have limited usefulness as animal models. Accordingly, there is still a need for a mutein of NTCP that renders cells or animals susceptible for an infection with HBV and Hepatitis D virus (HDV). The technical problem therefore is to comply with this need.

### SUMMARY OF THE INVENTION

The technical problem is solved by the subject-matter as defined in the claims. It is presented herein a NTCP mutein, a nucleic acid and a vector encoding the NTCP mutein, a host cell, a transgenic animal, methods for producing cells and animals susceptible for an infection with HBV and/or HDV. Also provided is a use of the host cell or the transgenic animal in a method for screening.

Accordingly, the present invention relates to a porcine sodium taurocholate cotransporter polypeptide (NTCP) mutein, wherein the mutein comprises glycine at sequence position 158, valine at sequence position 164, valine at sequence position 166 and leucine at sequence position 167 relative to the sequence positions of wild type porcine NTCP depicted as SEQ ID NO: 2. Optionally, the mutein further comprises an amino acid residue selected from the group consisting of lysine, arginine, glycine and valine at sequence position 157. The mutein may be obtained by genetic engineering of the wild type porcine sequence, e.g. from SEQ ID NO: 2.

Accordingly, the present invention also relates to a porcine sodium taurocholate cotransporter polypeptide (NTCP) mutein, wherein the mutein comprises lysine at sequence position 157, glycine at sequence position 158, valine at sequence position 164, valine at sequence position 166 and leucine at sequence position 167 relative to the sequence positions of wild type porcine NTCP depicted as SEQ ID NO: 2. The mutein may be obtained by genetic engineering of the wild type porcine sequence, e.g. from SEQ ID NO: 2.

The present invention also relates to a porcine sodium taurocholate cotransporter polypeptide (NTCP) mutein, the mutein comprising the sequence KGIVISLVLVL depicted as SEQ ID NO: 1 at sequence positions 157-167 or GIVISLVLVL depicted as SEQ ID NO: 11 at sequence positions 158-167 relative to the sequence positions of wild type porcine NTCP depicted as SEQ ID NO: 2. The mutein may be obtained by genetic engineering of the wild type porcine sequence, e.g. from SEQ ID NO: 2.

Preferably, the mutein is capable of rendering a cell genetically modified with the mutein being susceptible to an infection with hepatitis B virus (HBV) and/or hepatitis D virus (HDV) Preferably, the cell is porcine.

Preferably, the mutein has at least 82%, at least 85%, at least 90%, at least 95%, at least 98% or at least 99% sequence identity with SEQ ID NO: 3 or 12. Preferably, the mutein comprises SEQ ID NO: 3 or 12. Preferably, the mutein consists of a sequence as depicted in SEQ ID NO: 3 or 12.

The present invention also relates to a nucleic acid encoding the mutein.

The present invention also relates to a vector comprising the nucleic acid.

The present invention also relates to a host cell comprising at least one of the mutein, the nucleic acid and/or the vector.

Preferably, the host cell is not human. Preferably, the host cell is mammalian. Preferably, the cell is porcine. Preferably, the host cell is a hepatocyte.

The present invention also relates to a transgenic non-human animal comprising at least one of the mutein, of the nucleic acid, the vector, or the host cell. Preferably, the animal is a pig.

Preferably, the transgenic animal is susceptible to an infection with HBV and/or HDV. Preferably, the transgenic animal supports nuclear transport and entry of HBV or HDV.

The present invention also relates to a method for producing a cell, which is susceptible to HBV and/or HDV infection, the method comprising: (i) providing a cell, which is not susceptible to HBV and/or HDV infection, (ii) optionally disrupting endogenous copies of NTCP, (iii) genetically engineering the cell with the nucleic acid and/or the vector. Preferably, the cell is not human. Preferably, the cell is porcine.

The present invention also relates to a method for producing a cell, which is susceptible to HBV and/or HDV infection, the method comprising: (i) providing a cell, which is not susceptible to HBV and/or HDV infection, (ii) genetically modifying endogenous NTCP genes to comprise the sequence KGIVISLVLVL depicted as SEQ ID NO: 1 at sequence positions 157-167 or GIVISLVLVL depicted as SEQ ID NO: 11 at sequence positions 158-167 relative to the sequence positions of wild type porcine depicted as SEQ ID NO: 2. Preferably, the cell is not human. Preferably, the cell is porcine.

The present invention also relates to a method for producing a transgenic animal, the method comprising: (i) providing an animal, which is not susceptible to HBV and/or HDV infection, (ii) genetically modifying the endogenous NTCP genes to comprise the sequence KGIVISLVLVL depicted as SEQ ID NO: 1 at sequence positions 157-167 or GIVISLVLVL depicted as SEQ ID NO: 11 at sequence positions 158-167 relative to the sequence positions of wild type porcine mutein depicted as SEQ ID NO: 2; thereby rendering the animal susceptible to HBV and/or HDV infection. Preferably, the cell is not human. Preferably, the cell is porcine.

The present invention also relates to a use of the host cell or the transgenic animal in a method for screening for compounds, which prevent and/or treat an infection with HBV and/or HDV.

The present invention also relates to a use of the host cell of the invention or the transgenic animal of the invention for developing therapeutic strategies for treating an infection with HBV and/or HDV.

The present invention also relates to a use of the mutein for rendering a cell susceptible for an infection with HBV and/or HDV. Preferably, the cell is not human.

The present invention also relates to a method for identifying a compound, which is useful in the prevention and/or treatment of HBV and/or HDV infection, the method comprising: (i) providing the host cell or the transgenic animal; (ii) contacting the host cell or the transgenic animal with the compound to be tested.

The present invention also relates to a method for identifying a therapeutic strategy, which is useful in the prevention and/or treatment of HBV and/or HDV infection, the method comprising: (i) providing the host cell of the invention or the transgenic animal of the invention; (ii) subjecting the host cell or the transgenic animal to the therapeutic strategy to be tested.

### BRIEF DESCRIPTION OF THE DRAWINGS

The invention will be better understood with reference to the detailed description when considered in conjunction with the non-limiting examples and the accompanying drawings, in which:

FIG. 1 shows an alignment of human NTCP (SEQ ID NO: 4), the porcine NTCP (SEQ ID NO: 2) and the variant of the porcine NTCP used by the inventors (SEQ ID NO: 5). Sequence positions 157-167 are marked in grey and differences to the human NTCP are bold and underlined.

FIG. 2 shows the quantification of HBeAg in the supernatant of primary porcine hepatocytes (PPH) at day 3 and day 5 after infection with HBV. phNTCP is a mutein of porcine NTCP comprising amino acid sequence positions 157-167 of the human NTCP.

FIG. 3 shows a quantification of HBV rcDNA and cccDNA in PPH at day 5 after infection with HBV. phNTCP is a mutein of porcine NTCP comprising amino acid sequence positions 157-167 of the human NTCP.

FIG. 4 shows a Southern Blot analysis of HBV cccDNA in PPH at day 4 after infection with HBV. phNTCP is a mutein of porcine NTCP comprising amino acid sequence positions 157-167 of the human NTCP.

FIG. 5A shows the susceptibility of HepG2 cells transfected with different NTCP constructs to HBV. Here, the amount of HBV protein HBeAg in the supernatant of the cells is shown. HBeAg was quantified on day 4 (left bar), day 7 (middle bar) and day 10 (right bar).

FIG. 5B shows an alignment of different NTCP muteins. Also depicted is if the mutein comprises the human amino acid sequence positions 157-167 and if this mutein is enabling an HBV infection.

FIG. 6 shows the result of a sequencing of cell pools 1 and 2 after CRISPR/Cas9-mediated “humanization” of porcine NTCP.

FIG. 7 shows the strategy for “humanizing” sequence positions 157-167 in porcine NTCP.

### DETAILED DESCRIPTION OF THE INVENTION

The present invention is described in detail in the following and will also be further illustrated by the appended examples and figures.

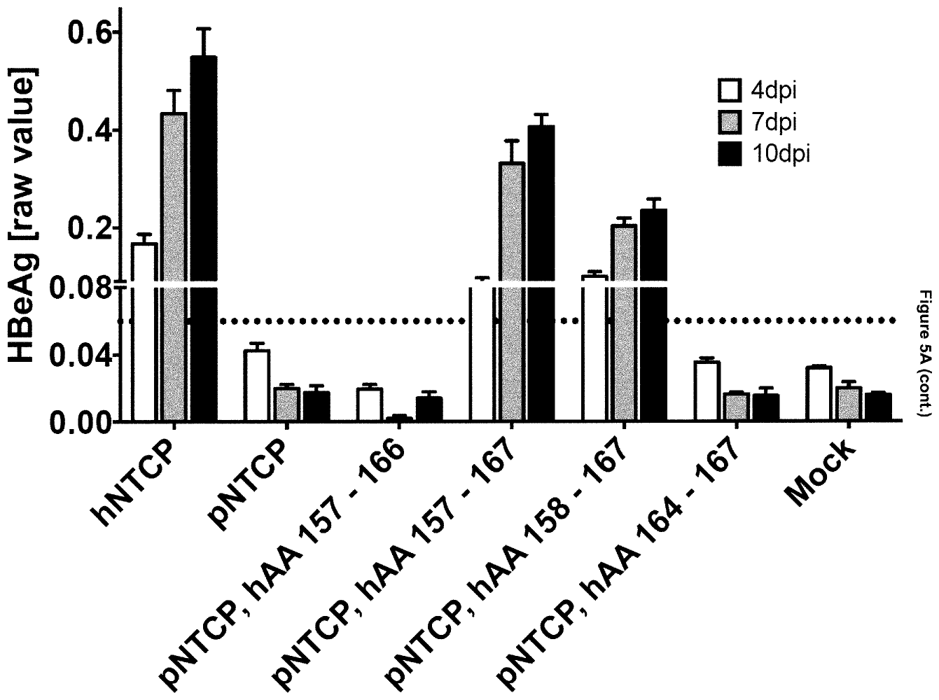

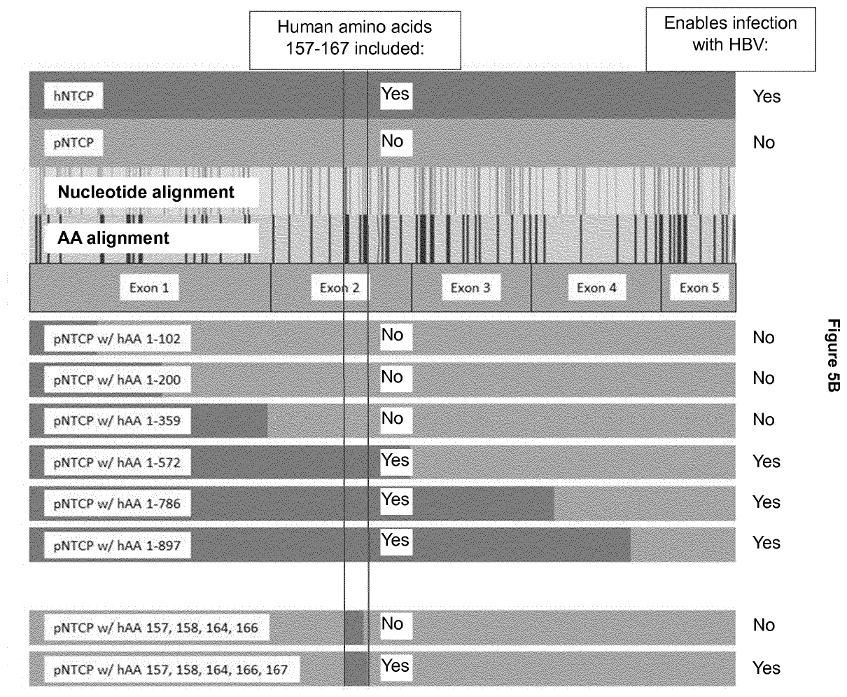

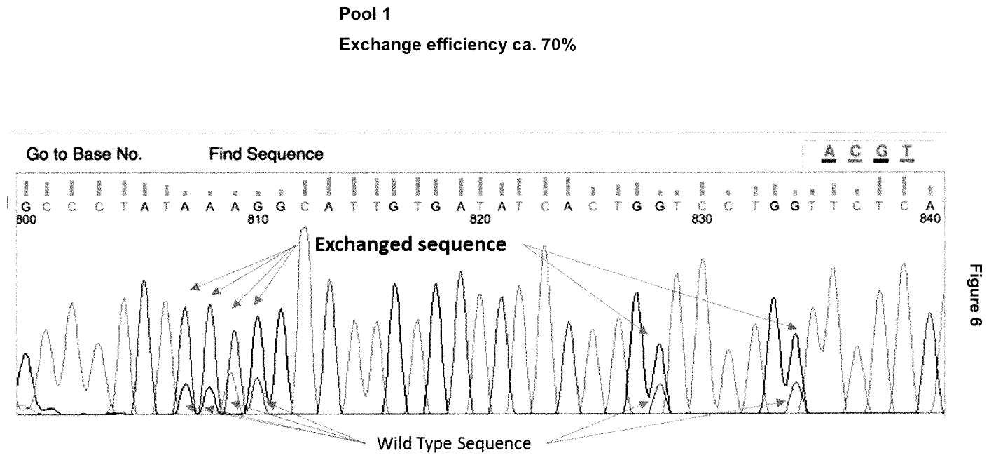

The inventors surprisingly found that it is not sufficient to “humanize” positions 157-165 of porcine NTCP as indicated in prior art, but instead positions 158-167 have to be humanized. Also, sequence positions 84-87 (H84R, T86K, and S87N) have been described to be essential for HBV binding to mouse NTCP (He et al., 2016). However, mice expressing this mutated mouse NTCP were still not susceptible for an HBV infection, presumably because of other cellular factors. In addition, it was shown in Old World monkeys that a mutation of the amino acids 157-165 (G157K, R158G, I160V, L161I, P165L) of the macaque NTCP (mcNTCP) renders those monkeys susceptible for an infection with HBV. However, neither of these approaches was successful with porcine NTCP (see Example 1 and FIG. 5).

As shown in Example 2, it is also necessary to introduce mutations at position 166 to valine and at sequence position 167 to leucine. This mutein of porcine NTCP (hpNTCP) is capable of making cells susceptible to an infection with HBV. In this context, the inventors made use of a porcine NTCP with a sequence that differs at position 167 from the published pNTCP sequence depicted in SEQ ID NO: 2. The sequence, which the inventors used for their experiment, contained the mutation L167P as shown in SEQ ID NO: 5. Thus, the inventors also found, using their inventive skills, that all the sequence positions 157-167 have to be identical to the human NTCP sequence. There is no incentive in prior art how porcine NTCP should have been mutated to enable binding of HBV and HDV and consequently enable infections with HBV and HDV.

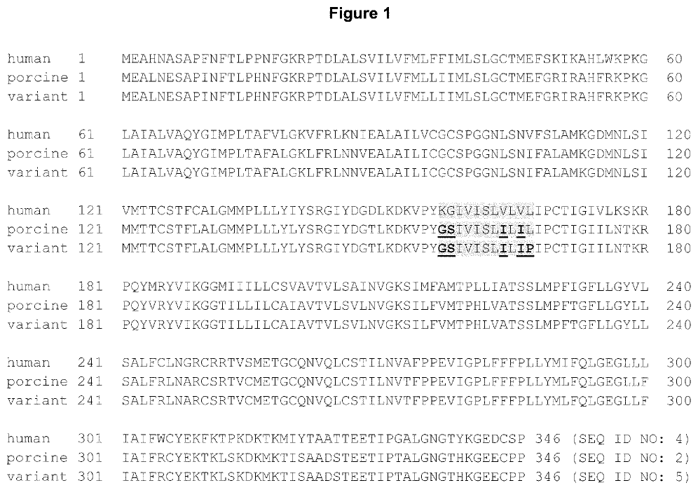

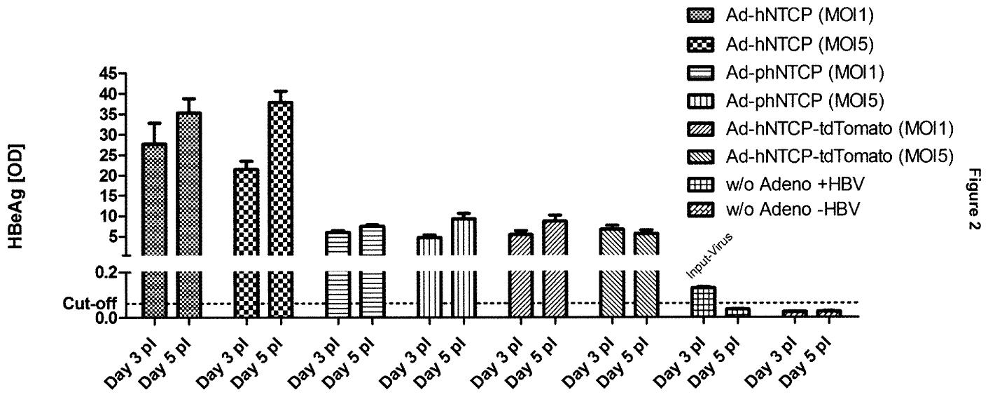

Positions 157, 158, 164, 166 and 167 (only in case of L167P mutation) differ from hNTCP as depicted in SEQ ID NO: 3 (see also the sequence alignment in FIG. 1), and positions 158, 164, 166 and 167 (only in case of L167P mutation) have to be mutated in pNTCP to generate a hpNTCP mutein that may render a cell susceptible to an infection with HBV and/or HDV. Position 157 may be “humanized” as well. Accordingly, the present invention relates in one embodiment to a porcine sodium taurocholate cotransporter polypeptide (NTCP) mutein, wherein the mutein comprises lysine at sequence position 157, glydne at sequence position 158, valine at sequence position 164, valine at sequence position 166 and leucine at sequence position 167 relative to the sequence positions of wild type porcine NTCP depicted as SEQ ID NO: 2.

However, the mutation at sequence position 157 is not necessary for entry of HBV and/or HDV into a cell. As shown in Müller et al. 2018, NTCPs from different monkey species vary at position 157 of NTCP. While this position varied and included lysine, arginine, glycine and valine as possible amino acids at sequence position 157, the variation had no influence on the susceptibility of human host cells to an HBV infection. Accordingly, position 157 of porcine NTCP may be any amino acid. In illustrative embodiments the amino acid residue at sequence position 157 may be selected from the group consisting of lysine, arginine, glycine and valine. Accordingly, the present invention relates to a porcine sodium taurocholate cotransporter polypeptide (NTCP) mutein, wherein the mutein comprises glycine at sequence position 158, valine at sequence position 164, valine at sequence position 166 and leucine at sequence position 167 relative to the sequence positions of wild type porcine NTCP depicted as SEQ ID NO: 2. An exemplary embodiment is shown in SEQ ID NO: 12.

NTCP, also known as sodium taurocholate cotransporter polypeptide or solute carrier family 10 member 1 (SLC10A1), binds as a cotransporter two sodium ions and one (conjugated) bile salt molecule, thereby providing an hepatic influx of bile salts. Other transported molecules include steroid hormones, thyroid hormones and various xenobiotics. NTCP is also the cell surface receptor necessary for the entry of HBV and HDV. In a preferred embodiment, the NTCP is porcine. A preferred NTCP is shown in SEQ ID NO: 2, which is identical to the UniProt Entry F1S4B1-1.

Hepatitis B is an infectious disease caused by the hepatitis B virus (HBV) that affects the liver. It can cause both acute and chronic infections. Many people have no symptoms during the initial infection. Some develop a rapid onset of sickness with vomiting, yellowish skin, tiredness, dark urine and abdominal pain. Often these symptoms last a few weeks and rarely does the initial infection result in death. In those who get infected around the time of birth, 90% develop chronic hepatitis B, while less than 10% of those infected after the age of five do. Most of those with chronic disease have no symptoms; however, cirrhosis and liver cancer may eventually develop. These complications result in the death of 15 to 25% of those with chronic disease.

Hepatitis D is a disease caused by the hepatitis D virus (HDV), a small spherical enveloped virusoid. HDV is considered to be a subviral satellite because it can propagate only in the presence of the hepatitis B virus (HBV). Transmission of HDV can occur either via simultaneous infection with HBV (coinfection) or superimposed on chronic hepatitis B or hepatitis B carrier state (superinfection). Both superinfection and coinfection with HDV results in more severe complications compared to infection with HBV alone. These complications include a greater likelihood of experiencing liver failure in acute infections and a rapid progression to liver cirrhosis, with an increased risk of developing liver cancer in chronic infections. In combination with hepatitis B virus, hepatitis D has the highest fatality rate of all the hepatitis infections, at 20%.

Both, HBV and HDV share the same envelope proteins and depend on NTCP for cell entry. Hence, a cell that is susceptible for HBV infection is also susceptible for HDV infection.

As used herein, a “mutein,” a “mutated” entity (whether protein or nucleic acid), or “mutant” refers to the exchange, deletion, or insertion of one or more nucleotides or amino acids, compared to the naturally occurring (wild-type) nucleic acid or protein “reference” scaffold. Said term may also include fragments of a mutein and variants as described herein. Porcine NTCP muteins of the present invention, fragments or variants thereof preferably have the function of making cells and/or animals susceptible to an infection with HBV and/or HDV. Within the context of the present invention, a mutein could also be described as a chimeric NTCP.

Preferably, the NTCP mutein of the present invention is obtained by genetic engineering of the wild type porcine sequence, e.g. from SEQ ID NO: 2.

“Fragment” as used herein describes a part of a certain nucleotide sequence, gene, amino acid sequence or a protein. Such a fragment may be shortened by at least one amino acid at N-terminal or C-terminal of a protein or at least one nucleotide at the 5′- or 3′-end of a polynucleotide. A fragment of a mutein of the invention may lack 1, 2, 3, 4, 5, 6, 7, 8, 9, 10, 15, 20, 25, 30, 35 or 40 amino acids at the N-terminus and/or C-terminus. A fragment of a polynucleotide encoding a mutein of the invention may lack 1, 2, 3, 4, 5, 6, 7, 8, 9, 10, 15, 20, 25, 30, 35 or 40 nucleotides at the 5′ end and/or the 3′ end. An important feature of the fragments is that they retain their ability to carry out the activity of the source sequence, i.e. may render cells susceptible for an infection with HBV and/or HDV.

The term “variant” as used herein relates to derivatives or variants of a protein or peptide that include modifications of the amino acid sequence, for example by substitution, deletion, insertion or chemical modification. Such variants include proteins, wherein one or more amino acids have been replaced by their respective D-stereoisomers or by amino acids other than the naturally occurring 20 amino acids, such as, for example, ornithine, hydroxyproline, citrulline, homoserine, hydroxylysine, norvaline, or wherein one or more amino acid residues are conservatively substituted compared to said polypeptide. A “conservative substitution” as used herein is an amino acid substitution that changes an amino acid to a different amino acid with similar biochemical properties (e.g. charge, hydrophobicity and size). Examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan.

In another embodiment, the present invention relates to a porcine NTCP mutein comprising the sequence KGIVISLVLVL depicted as SEQ ID NO: 1 at sequence positions 157-167 relative to the sequence positions of wild type porcine NTCP depicted as SEQ ID NO: 2. This porcine NTCP mutein comprises the essential part of human NTCP. In a further embodiment, the present invention relates to a porcine NTCP mutein comprising the sequence GIVISLVLVL depicted as SEQ ID NO: 11 at sequence positions 158-167 relative to the sequence positions of wild type porcine NTCP depicted as SEQ ID NO: 2.

As shown in Example 2, a cell is susceptible to infection with HBV, if the porcine NTCP mutein of the invention is expressed. As outlined herein, susceptibility for HBV translates to susceptibility for HDV. Accordingly, the mutein of the invention is preferably capable of rendering a cell genetically modified with the mutein being susceptible to an infection with hepatitis B virus (HBV) and/or hepatitis D virus (HDV), wherein the cell preferably is porcine. In one embodiment, the mutein of the invention is preferably capable of rendering a cell genetically modified with the mutein being susceptible to an infection with hepatitis B virus (HBV), wherein the cell is preferably porcine. In another embodiment, the mutein of the invention preferably is capable of rendering a cell genetically modified with the mutein being susceptible to an infection with hepatitis D virus (HDV), wherein the cell is preferably porcine.

SEQ ID NO: 3 shows an exemplary embodiment of the mutein of the invention. Here, the porcine NTCP as depicted in SEQ ID NO: 2 has been modified with the sequence KGIVISLVLVL depicted as SEQ ID NO: 1. In one embodiment, the mutein has at least 82%, at least 85%, at least 90%, at least 95%, at least 98% or at least 99% sequence identity with SEQ ID NO: 3. In another embodiment, the mutein comprises SEQ ID NO: 3. In a further embodiment, the mutein consists of the sequence depicted in SEQ ID NO: 3. Human (SEQ ID NO: 4) and porcine (SEQ ID NO: 2) share a sequence identity of only 81%. However, as shown by the inventors, it is sufficient to “humanize” positions 157-167 or even only positions 158-167 of the porcine NTCP. Thus, crucial for the function as HBV and/or HDV receptor are positions 157/158-167. This means, sequence variations in other parts of the NTCP are allowed and do not impair the function of NTCP as entry receptor for HBV/HDV. Even sequence identities as low as 80% to SEQ ID NO: 3 or 12 still encompass muteins of porcine NTCP that render a cell genetically modified with the mutein susceptible to an infection with hepatitis B virus (HBV) and/or hepatitis D virus (HDV). 81% sequence identity in this context means that around 66 amino acids may be substituted without having any effect on the function of rendering a (porcine) cell susceptible for an infection with HBV and/or HDV.

Preferably, the mutein has at least 82%, at least 85%, at least 90%, at least 95%, at least 98% or at least 99% sequence identity with SEQ ID NO: 3, more preferably the mutein still includes the required substitutions, i.e. preferably comprises glycine at sequence position 158, valine at sequence position 164, valine at sequence position 166 and leucine at sequence position 167 relative to the sequence positions of wild type porcine NTCP depicted as SEQ ID NO: 2, more preferably comprising additionally an amino acid residue selected from the group consisting of lysine, arginine, glycine and valine at sequence position 157 and most preferably position 157 is lysine.

SEQ ID NO: 12 is an exemplary embodiment of a NTCP mutein that is “humanized” at positions 158-167. Accordingly, in one embodiment, the mutein has at least 82%, at least 85%, at least 90%, at least 95%, at least 98% or at least 99% sequence identity with SEQ ID NO: 12, more preferably the mutein still includes the required substitutions, i.e. preferably comprises glycine at sequence position 158, valine at sequence position 164, valine at sequence position 166 and leucine at sequence position 167 relative to the sequence positions of wild type porcine NTCP depicted as SEQ ID NO: 2, more preferably comprising additionally an amino acid residue selected from the group consisting of lysine, arginine, glycine and valine at sequence position 157 and most preferably position 157 is lysine. In another embodiment, the mutein comprises SEQ ID NO: 12. In a further embodiment, the mutein consists of the sequence depicted in SEQ ID NO: 12.

The term “homology” as used herein in its usual meaning and includes identical amino acids as well as amino acids which are regarded to be conservative substitutions (for example, exchange of a glutamate residue by an aspartate residue) at equivalent positions in the linear amino acid sequence of two proteins that are compared with each other. By “identity” or “sequence identity” is meant a property of sequences that measures their similarity or relationship. The term “sequence identity” or “identity” as used in the present invention means the percentage of pair-wise identical residues—following (homology) alignment of a sequence of a polypeptide of the invention with a sequence in question—with respect to the number of residues in the longer of these two sequences. Identity is measured by dividing the number of identical residues by the total number of residues and multiplying the product by 100.

The percentage of sequence homology or sequence identity can, for example, be determined herein using the program BLASTP, version blastp 2.2.5 (Nov. 16, 2002; cf. Altschul, S. F. et al. (1997) Nucl. Acids Res. 25, 3389-3402). In this embodiment the percentage of homology is based on the alignment of the entire polypeptide sequences (matrix: BLOSUM 62; gap costs: 11.1; cutoff value set to 10⁻³) optionally including the propeptide sequences, using the human IL-4 as reference in a pairwise comparison. It is calculated as the percentage of numbers of “positives” (homologous amino acids) indicated as result in the BLASTP program output divided by the total number of amino acids selected by the program for the alignment. It is noted in this connection that this total number of selected amino acids can differ from the length of the porcine NTCP.

For expression of the porcine NTCP mutein a person skilled in the art may use a nucleic acid encoding the porcine NTCP mutein. Accordingly, tie present invention also relates to a nucleic acid encoding the NTCP mutein of the invention. Preferably, the nucleic acid encoding the NTCP mutein of the invention is DNA. However, also overexpression of the NTCP mutein of the invention using RNA is envisioned.

In some embodiments, a nucleic acid molecule, such as DNA, disclosed in this application may be “operably linked” to another nucleic acid molecule of the disclosure to allow expression of a porcine NTCP mutein of the disclosure. In this regard, an operable linkage is a linkage in which the sequence elements of the first nucleic acid molecule and the sequence elements of the second nucleic acid molecule are connected in a way that enables expression of the fusion protein as a single polypeptide.

Preferably, the nucleic acid is comprised in a vector. Accordingly, the present invention also relates to a vector comprising the nucleic acid of the invention. Preferably, the vector is for the expression of the porcine NTCP mutein in a host cell. This vector may additionally comprise a promotor to allow the overexpression of the polynucleotide in a host cell, a selection marker, to enrich host cells comprising the vector. In a vector, the nucleic acid encoding the porcine NTCP mutein is preferably operatively linked to a promotor. Preferably, the vector is an adenoviral vector. Exemplary embodiments for pNTCP muteins comprised in an adenoviral vector are shown in SEQ ID NOs: 6-10.

As described herein, a cell comprising the muteins of the inventions may be susceptible for an infection with HBV and/or HDV. Hence, cells or host cells comprising the mutein of the invention are very promising tools for research. Accordingly, the present invention relates to a host cell comprising at least one of the mutein of the invention, the nucleic acid of the invention and/or the vector of the invention. In one embodiment, the host cell does not comprise any other or endogenous NTCP except for the porcine NTCP mutein of the invention. In a further embodiment, the host cell is not human. Preferably, the host cell is mammalian, more preferably porcine. In a further preferred embodiment, the host cell is a hepatocyte. Hepatocytes are the main cell type that is infected by HBV and/or HDV. In one embodiment, the host cell is not a mouse. In one embodiment, the host cell is not a member of the family of Muridae. In another embodiment, the host cell is not a member of the Family of Cercopithecidae.

As shown in Example 2, primary porcine hepatocytes transduced with the mutein of the invention are susceptible to an infection with HBV and/or HDV. While primary cell culture systems already allow in vitro experiments, a transgenic animal is needed for in vivo experiments. Accordingly, the present invention relates to a transgenic animal comprising at least one of the muteins of the invention, the nucleic acid of the invention, the vector of the invention and/or the host cell of the invention. Animals may include, but are not limited to species, which have already been proven amenable to genetic modification, such as sheep, goat, cow, pig, dog, non-human primates. Preferably, the animal is non-human. More preferably, the animal is a pig. In one embodiment, the animal is not a mouse. In one embodiment, the animal is not a member of the family of Muridae. In another embodiment, the animal is no member of the Family of Cercopithecidae, particularly no macaque. The transgenic animal is preferably susceptible to an infection with HBV and/or HDV. Other preferred examples for the animal are Gairdner's shrewmouse, bat, Bactrian camel, dromedary, alpaca, marmot, donkey, cheetah, tiger, rhinoceros, deer or cat.

The term “the transgenic animal is susceptible to an infection with HBV and/or HDV” means, for example, that the transgenic animal supports nuclear transport and entry of HBV or HDV. In general, a cell or an animal may be described as “susceptible to an infection with HBV and/or” if it can be infected by HBV and/or HDV. A person skilled in the art is aware of assays to determine whether a cell or an animal has been infected by HBV and/or HDV or not, i.e. whether the cell or animal is susceptible to an infection with HBV and/or HDV or not. Such assays include, for example, the detection of viral nucleic acids such as the viral cccDNA by Southern Blot and/or quantitative PCR, or viral proteins such as HBeAg by ELISA or Western Blot that have been used in the Examples.

The present invention also relates to a method for producing a cell, which is susceptible to HBV and/or HDV infection. Within such a method, optionally (all) endogenous copies of NTCP may be disrupted so that all NTCP expressed in the cell are the NTCP muteins of the invention. The cell may be genetically engineered with the nucleic acid or the vector of the invention. Accordingly, the present invention relates to a method for producing a cell, which is susceptible to HBV and/or HDV infection, the method comprising: (i) providing a cell, which is not susceptible to HBV and/or HDV infection, (ii) optionally disrupting endogenous copies of NTCP, (iii) genetically engineering the cell with the nucleic acid and/or the vector of the invention. Preferably, the cell is not human. More preferably, the cell is porcine. Other preferred examples for the cell are Gairdner's shrewmouse, bat, Bactrian camel, dromedary, alpaca, marmot, donkey, cheetah, tiger, rhinoceros, deer or cat.

Another possible method to produce a cell, which is susceptible to HBV and/or HDV infection, is based on the genetic modification of endogenous NTCP genes. Within this embodiment of a method for producing a cell, which is susceptible to HBV and/or HDV infection, the method comprises: (i) providing a cell, which is not susceptible to HBV and/or HDV infection, (ii) genetically modifying endogenous NTCP genes to comprise the sequence KGIVISLVLVL depicted as SEQ ID NO: 1 at sequence positions 157-167 relative to the sequence positions of wild type porcine depicted as SEQ ID NO: 2. Preferably, the cell is not human. More preferably, the cell is porcine. Other preferred examples for the cell are Gairdner's shrewmouse, bat, Bactrian camel, dromedary, alpaca, marmot, donkey, cheetah, tiger, rhinoceros, deer and cat.

The present invention further relates to a method for producing a transgenic animal. Here, an animal, which is not susceptible to HBV and/or HDV infection is genetically modified to comprise the sequence KGIVISLVLVL (SEQ ID NO: 1) at sequence positions 157-167 in the endogenous NTCP genes. Such a transgenic animal is susceptible to HBV and/or HDV infection. Accordingly, the present invention relates to a method for producing a transgenic animal, the method comprising: (i) providing an animal, which is not susceptible to HBV and/or HDV infection, (ii) genetically modifying the endogenous NTCP genes to comprise the sequence KGIVISLVLVL depicted as SEQ ID NO: 1 at sequence positions 157-167 relative to the sequence positions of wild type porcine mutein depicted as SEQ ID NO: 2; thereby rendering the animal susceptible to HBV and/or HDV infection. Preferably, the animal is not human. More preferably, the animal is porcine. Other preferred examples for the animal are Gairdner's shrewmouse, bat, Bactrian camel, dromedary, alpaca, marmot, donkey, cheetah, tiger, rhinoceros, deer and cat.

In an alternative embodiment, the present invention relates to a method for producing a transgenic animal, the method comprising: (i) providing an animal, which is not susceptible to HBV and/or HDV infection, (ii) optionally disrupting endogenous copies of NTCP, (iii) genetically engineering the animal with the nucleic acid and/or the vector of the invention and/or genetically modifying the endogenous NTCP genes to comprise the sequence KGIVISLVLVL depicted as SEQ ID NO: 1 at sequence positions 157-167 relative to the sequence positions of wild type porcine NTCP depicted as SEQ ID NO: 2; thereby rendering the animal susceptible to HBV and/or HDV infection.

To elucidate whether a host cell or animal has been successfully genetically modified, a fluorescence-labeled Myrcludex B may be used because it only binds to human or chimeric and not porcine NTCP. Alternatively, the DNA of the host cell or animal of interest could be sequenced, or a PCR/qPCR could be applied with primers that only allow amplification of DNA isolated from the host cell, which comprises the genetic modification, i.e. comprises SEQ ID NO: 1 at sequence positions 157-167 relative to the sequence positions of wild type porcine NTCP as depicted in SEQ ID NO: 2. Further ways to verify a successful genetic modification are known to a person skilled in the art.

A person skilled in the art is capable of disrupting and/or modifying endogenous NTCP genes. Within this context, “disrupting endogenous copies” relates to reducing the number of the endogenous NTCP genes in a host cell or transgenic animal and/or to mutate the endogenous NTCP genes comprised in a host cell or animal to prevent its translation or expression. Said disruption can be achieved by destroying the genetic information encoding the polynucleotide in the genome or plasmid of the host cell or transgenic animal by molecular and/or genetic engineering. Molecular and/or genetic engineering methods include, but are not limited to, gene editing such as the use of targeting endonucleases or gene targeting. The nucleases create specific double-stranded chromosomal breaks (DSBs) at desired locations in the genome, which in some cases harnesses the cell's endogenous mechanisms to repair the induced break by natural processes of homologous recombination (HR) and/or non-homologous end-joining (NHEJ). Gene editing effectors include Zinc Finger Nucleases (ZFNs), Transcription Activator-Like Effector Nucleases (TALENs), the Clustered Regularly Interspaced Short Palindromic Repeats/CAS9 (CRISPR/Cas9) system, and meganucleases (e.g., meganucleases re-engineered as homing endonucleases). “Genetically modifying” relates to the modification of a gene already present in the host cell or animal. Methods for genetic modification include, but are not limited to, gene targeting or gene editing. These nucleases outlined herein may also be used for site-specific mutations of endogenous NTCP copies, i.e. for modifying the endogenous NTCP genes to comprise the sequence KGIVISLVLVL depicted as SEQ ID NO: 1 at sequence positions 157-167 relative to the sequence positions of wild type porcine mutein depicted as SEQ ID NO: 2—a process also known as gene editing.

In detail, a double-strand break may be induced in exon 2 of the NTCP gene by e.g. CRISPR/Cas9 or TALEN. Using a vector or direct transfection, a DNA and/or RNA molecule with sequences that are homologous to the adjacent intron sequences can be introduced in the host cell or animal. This DNA or RNA molecule comprises the modified gene sequence such as the sequence KGIVISLVLVL depicted as SEQ ID NO: 1 at sequence positions 157-167. During homologous end-joining there is a chance that the introduced DNA or RNA molecule will be used as template and thereby the genomic DNA is modified. This DNA or RNA molecule may also comprise a resistance cassette comprising a promoter, a resistance gene and a polyA signal upstream or downstream the transgenic Exon 2, to enable a subsequent selection. This procedure is preferably performed in an ovum (cell), which can be implanted in an animal such as a pig after successful completion. Alternatively, the nucleus of a transgenic non-pluripotent cell could be transferred into a fertilized and denucleated ovum (cell) and then implanted into the animal.

Genetic modification or modifications of a cell or an animal might include the introduction of the NTCP mutein into the animal or cell alone or in conjunction with other elements lacking in the animal or cell to be modified.

Complying with the need for new and improved medicaments for the treatment of HBV and/or HDV can also be achieved using the NTCP muteins of the present invention. The NTCP muteins of the invention may be used in screening for compounds, which prevent and/or treat an infection with HBV and/or HDV. Accordingly, the present invention relates to the use of a host cell of the invention or a transgenic animal of the invention in a method for screening for compounds, which prevent and/or treat an infection with HBV and/or HDV.

A method for screening or identifying a compound, which is useful in the prevention and/or treatment of HBV and/or HDV infection, may comprise providing the host cell or the transgenic animal of the invention and contacting the host cell or the transgenic animal of the invention with the compound to be tested. To enable a decision whether a compound is effective or not, the host cell or the transgenic animal is infected with HBV and/or HDV after contacting the host cell or the transgenic animal with the compound to be tested. Effective compounds can be identified by comparison with non-treated or placebo-treated (group of) host cell(s) or transgenic animal(s). Accordingly, the present invention relates to a method for identifying a compound, which is useful in the prevention and/or treatment of HBV and/or HDV infection, the method comprising: (i) providing the host cell or the transgenic animal of the invention; (ii) contacting the host cell or the transgenic animal with the compound to be tested. The method for screening or identifying a compound, which is useful in the prevention and/or treatment of HBV and/or HDV infection, may further comprise a step of detecting HBV and/or HDV infection in the host cell or the transgenic animal of the invention.

Examples for “compounds” that can be tested include proteins, peptides and small molecules.

The compound to be tested can e.g. be an “antibody molecule”. An “antibody molecule” as used herein can be a full length antibody, a recombinant antibody molecule, or a fully human antibody molecule. A full length antibody is any naturally occurring antibody. The term “antibody” also includes immunoglobulins (Ig's) of different classes (i.e. IgA, IgG, IgM, IgD and IgE) and subclasses (such as IgG1, IgG2 etc.). Such full length antibodies can be isolated from different animals such as e.g. different mammalian species. A “recombinant antibody molecule” refers to an antibody molecule the genes of which has been cloned, and that is produced recombinantly in a host cell or organism, using well-known methodologies of genetic engineering. Typically, a recombinant antibody molecule has been genetically altered to comprise an amino acid sequence, which is not found in nature. Thus, a recombinant antibody molecule can be a chimeric antibody molecule or a humanized antibody molecule. In preferred embodiments, the fusion protein comprises the heavy chain of an immunoglobulin described herein and an IL-15 mutant described herein, which may be connected via a linker described herein. In this arrangement, it is preferred that the immunoglobulin moiety is located N terminally of the IL-15 mutant. In such a fusion protein, the light chain of the antibody molecule is paired with the antibody heavy chain as in any regular antibody or antibody fragment.

The compound to be tested can also be an “antibody fragment”. Such antibody fragments comprise at least those parts of an antibody, that form the (antigen) binding site. Illustrative examples of such an antibody fragment are single chain variable fragments (scFv), Fv fragments, single domain antibodies, such as e.g. VHH (camelid) antibodies, di-scFvs, fragment antigen binding regions (Fab), F(ab′)₂ fragments, Fab′ fragments, diabodies, domain antibodies, (Holt L J, Herring C, Jespers L S, Woolven B P, Tomlinson I M. Domain antibodies: proteins for therapy. Trends Biotechnol. 2003 November; 21(11): 484-90), or bispecific “Fabsc”-antibody molecules as described in International patent application WO 2013/092001 comprising a single chain Fv fragment, which is connected to an Fab fragment via a CH2 domain to name only a few.

The compound to be tested can also be a proteinaceous binding molecule with antibody-like binding properties. Illustrative examples of proteinaceous binding molecules with antibody like binding properties that can be used as binding proteins include, but are not limited to, an aptamer, a mutein based on a polypeptide of the lipocalin family (exemplary lipocalin muteins that are also known under their trademark name “Anticalin®” are, for example, described in PCT applications WO 99/16873, WO 00/75308, WO 03/029471, WO 03/029462, WO 03/029463, WO 2005/019254, WO 2005/019255, WO 2005/019256, WO 200656464 or WO 2008/015239, or the review article of Skerra, A. (2001) Rev. Mol. Biotechnol. 74, 257-275), a glubody, a protein based on the ankyrin scaffold, a protein based on the crystalline scaffold, an adnectin, an avimer, a EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain (complement control protein (CCP) modules), a Link domain, a Thrombospondin type I domain, an immunoglobulin domain or a an immunoglobulin-like domain (for example, domain antibodies or camel heavy chain antibodies), a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, “Kappabodies” (III CR1, Gonzales J N, Houtz E K, Ludwig J R, Melcher E D, Hale J E, Pourmand R, Keivens V M, Myers L, Beidler K, Stuart P, Cheng S, Radhakrishnan R. Design and construction of a hybrid immunoglobulin domain with properties of both heavy and light chain variable regions. Protein Eng. 1997 August; 10(8): 949-57) “Minibodies” (Martin F I, Toniatti C, Salvati A L, Venturini S, Ciliberto G, Cortese R, Sollazzo M. The affinity-selection of a minibody polypeptide inhibitor of human interleukin-6. EMBO J. 1994 Nov. 15; 13(22): 5303-9), “Janusins” (Traunecker A, Lanzavecchia A, Karjalainen K. Bispecific single chain molecules (Janusins) target cytotoxic lymphocytes on HIV infected cells. EMBO J. 1991 December; 10(12): 3655-9 and Traunecker A, Lanzavecchia A, Karjalainen K. Janusin: new molecular design for bispecific reagents. Int J Cancer Suppl. 1992; 7: 51-2), a nanobody, an adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein, an avimer (Silverman J, Liu Q, Bakker A, To W, Duguay A, Alba B M, Smith R, Rivas A, Li P, Le H, Whitehorn E, Moore K W, Swimmer C, Perlroth V, Vogt M, Kolkman J, Stemmer W P. Multivalent avimer proteins evolved by exon shuffling of a family of human receptor domains. Nat Biotechnol. 2005 December; 23(12): 1556-61. Epub 2005 Nov. 20); as well as multivalent avimer proteins evolved by exon shuffling of a family of human receptor domains as also described in Silverman et al. (Silverman J, Liu Q, Bakker A, To W, Duguay A, Alba B M, Smith R, Rivas A, Li P, Le H, Whitehorn E, Moore K W, Swimmer C, Perlroth V, Vogt M, Kolkman J, Stemmer W P. Multivalent avimer proteins evolved by exon shuffling of a family of human receptor domains. Nat Biotechnol. 2005 December; 23(12): 1556-61. Epub 2005 Nov. 20).

Further, the compound to be tested can also make use of RNA interference. RNA interference (RNAi) is a biological process in which RNA molecules inhibit gene expression or translation, by neutralizing targeted mRNA molecules. Two types of small ribonucleic acid (RNA) molecules—microRNA (miRNA) and small interfering RNA (siRNA)—are central to RNA interference. The RNAi pathway is found in many eukaryotes, including animals, and is initiated by the enzyme Dicer, which cleaves long double-stranded RNA (dsRNA) molecules into short double-stranded fragments of ˜21 nucleotide siRNAs. Each siRNA is unwound into two single-stranded RNAs (ssRNAs), the passenger strand and the guide strand. The passenger strand is degraded and the guide strand is incorporated into the RNA-induced silencing complex (RISC). The most well-studied outcome is post-transcriptional gene silencing, which occurs when the guide strand pairs with a complementary sequence in a messenger RNA molecule and induces cleavage by Argonaute 2 (Ago2), the catalytic component of the RISC. In some organisms, this process spreads systemically, despite the initially limited molar concentrations of siRNA. Accordingly, the compound to be tested can be a miRNA or a siRNA. These miRNA or siRNA preferably is designed to deplete mRNA encoding for a viral protein.

The compound to be tested may also be or comprise a small molecule. Examples include nucleosides or derivatives thereof, nucleotides or derivatives thereof. Further, the compound to be tested could be an immune-modulatory or immune-stimulating compound such as an interferon or a derivative thereof. Interferon α is already used for therapy of HBV infections and the uses and methods of the invention provide the opportunity to test new derivatives in an animal model.

The compounds to be tested may have different modes of actions. Exemplary modes of actions that a compound to be tested may have include inhibitors of viral processes and/or inhibitors of processes of the host cell or the animal, which are essential for viral replication. Inhibition of viral processes may relate to inhibitors of viral entry, inhibitors of viral replication, inhibitors of virus assembly and/or inhibitors of viral release/budding. HBV and HDV rely on NTCP for viral entry. E.g., a compound that prevents binding to NTCP is an inhibitor of viral entry. Nucleotide analogues like lamivudine or telbivudin may prevent viral replication. As HBV uses its own viral RNA-dependent-DNA-polymerase, the viral RNA-dependent-DNA-polymerase may be an interesting target. Alternatively or additionally, the compound to be tested may inhibit pathways of the host cell or the animal on which HBV and/or HDV relies for the completion of its life cycle.

Additionally, the present invention relates to the use of a host cell of the invention or a transgenic animal of the invention for developing therapeutic strategies for treating an infection with HBV and/or HDV. Using “compounds” to treat and/or prevent HBV and/or HDV infections is not the only possible treatment and/or prophylaxis of HBV and/or HDV. Thus, also other therapeutic strategies can be tested for their efficacy and/or safety in treating and/or preventing HBV and/or HDV. Such other therapeutic strategies may include an immune-modulating or immune-activating therapy. Examples for such immune-modulating or immune-activating therapies include T cell redirection, therapeutic vaccination, activation of pattern recognition receptors, antibody-based therapies and the like.

A method for screening or identifying a therapeutic strategy, which is useful in the prevention and/or treatment of HBV and/or HDV infection, may comprise providing the host cell or the transgenic animal of the invention and subjecting the host cell or the transgenic animal of the invention to the therapeutic strategy to be tested. To enable a decision whether a therapeutic strategy is effective or not, the host cell or the transgenic animal is infected with HBV and/or HDV after subjecting the host cell or the transgenic animal to the therapeutic strategy to be tested. Effective therapeutic strategies can be identified by comparison with non-treated or placebo-treated (group of) host cell(s) or transgenic animal(s). Accordingly, the present invention relates to a method for identifying a therapeutic strategy, which is useful in the prevention and/or treatment of HBV and/or HDV infection, the method comprising: (i) providing the host cell of the invention or the transgenic animal of the invention; (ii) subjecting the host cell or the transgenic animal to the therapeutic strategy to be tested. The method for screening or identifying a therapeutic strategy, which is useful in the prevention and/or treatment of HBV and/or HDV infection, may further comprise a step of detecting HBV and/or HDV infection in the host cell or the transgenic animal of the invention.

As described herein, the muteins of the invention may be used to render a cell susceptible for an infection with HBV and/or HDV. Accordingly, the present invention relates to the use of a mutein of the invention for rendering a cell susceptible for an infection with HBV and/or HDV. Preferably, the cell is not human. Such a cell susceptible for an infection with HBV and/or HDV may be useful for identifying compounds or therapeutic strategies useful in treatment and/or prophylaxis of HBV and/or HDV.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

The invention is also characterized by the following items:

1. A porcine sodium taurocholate cotransporter polypeptide (NTCP) mutein, wherein the mutein comprises lysine at sequence position 157, glycine at sequence position 158, valine at sequence position 164, valine at sequence position 166 and leucine at sequence position 167 relative to the sequence positions of wild type porcine NTCP depicted as SEQ ID NO: 2.

2. A porcine sodium taurocholate cotransporter polypeptide (NTCP) mutein, the mutein comprising the sequence KGIVISLVLVL depicted as SEQ ID NO: 1 at sequence positions 157-167 relative to the sequence positions of wild type porcine NTCP depicted as SEQ ID NO: 2.

3. The mutein of item 1 or 2, wherein the mutein is capable of rendering a cell genetically modified with the mutein being susceptible to an infection with hepatitis B virus (HBV) and/or hepatitis D virus (HDV).

4. The mutein of item 3, wherein the cell is porcine.

5. The mutein of any of items 1 to 4, having at least 82%, at least 85%, at least 90%, at least 95%, at least 98% or at least 99% sequence identity with SEQ ID NO: 3.

6. The mutein of any one of items 1 to 4, comprising SEQ ID NO: 3.

7. The mutein of any one of items 1 to 6, consisting of a sequence as depicted in SEQ ID NO: 3.

8. A nucleic acid encoding the mutein of any one of items 1 to 7.

9. A vector comprising the nucleic acid of item 8.

10. A host cell comprising at least one of the mutein of any one of items 1 to 7, the nucleic acid of item 8 and/or the vector of item 9.

11. The host cell of item 10, wherein the host cell is not human.

12. The host cell of item 10 or 11, wherein the host cell is mammalian.

13. The host cell of any one of items 10 to 12, wherein the host cell is porcine.

14. The host cell of any one of items 10 to 14, wherein the host cell is a hepatocyte.

15. A transgenic non-human animal comprising at least one of the mutein of any one of items 1 to 7, the nucleic acid of item 8, the vector of item 9, or the host cell of any one of items 10 to 14.

16. The transgenic non-human animal of item 15, wherein the animal is a pig.

17. The transgenic non-human animal of item 15 or 16, wherein the transgenic animal is susceptible to an infection with HBV and/or HDV.

18. A method for producing a cell, which is susceptible to HBV and/or HDV infection, the method comprising:

(i) providing a cell, which is not susceptible to HBV and/or HDV infection,
(ii) optionally disrupting endogenous copies of NTCP,
(iii) genetically engineering the cell with the nucleic acid of item 8 and/or the vector of item 9.

19. A method for producing a cell, which is susceptible to HBV and/or HDV infection, the method comprising:

(i) providing a cell, which is not susceptible to HBV and/or HDV infection,
(ii) genetically modifying endogenous NTCP genes to comprise the sequence KGIVISLVLVL depicted as SEQ ID NO: 1 at sequence positions 157-167 relative to the sequence positions of wild type porcine depicted as SEQ ID NO: 2.

20. The method of item 18 or 19, wherein the cell is not human.

21. The method of any one of items 18 to 20, wherein the cell is porcine.

22. A method for producing a transgenic animal, the method comprising:

(i) providing an animal, which is not susceptible to HBV and/or HDV infection,
(ii) genetically modifying the endogenous NTCP genes to comprise the sequence KGIVISLVLVL depicted as SEQ ID NO: 1 at sequence positions 157-167 relative to the sequence positions of wild type porcine mutein depicted as SEQ ID NO: 2;
thereby rendering the animal susceptible to HBV and/or HDV infection.

23. The method of item 22, wherein the animal is not human.

24. The method of item 22 or 23, wherein the non-human animal is a pig.

25. Use of a host cell of any one of items 10 to 14 or a transgenic animal of any one of items 15 to 17 in a method for screening for compounds, which prevent and/or treat an infection with HBV and/or HDV.

26. Use of a host cell of any one of items 10 to 14 or a transgenic animal of any one of items 15 to 17 for developing therapeutic strategies for treating an infection with HBV and/or HDV.

27. Use of the mutein of any one of items 1 to 7 for rendering a cell susceptible for an infection with HBV and/or HDV.

28. Use of item 27, wherein the cell is not human.

29. A method for identifying a compound, which is useful in the prevention and/or treatment of HBV and/or HDV infection, the method comprising:

(i) providing the host cell of any one of items 10 to 14 or the transgenic animal of anyone of items 15 to 17;
(ii) contacting the host cell or the transgenic animal with the compound to be tested.

30. A method for identifying a therapeutic strategy, which is useful in the prevention and/or treatment of HBV and/or HDV infection, the method comprising:

(i) providing the host cell of any one of items 10 to 14 or the transgenic animal of anyone of items 15 to 17;
(ii) subjecting the host cell or the transgenic animal to the therapeutic strategy to be tested.

### EXAMPLES

An even better understanding of the present invention and of its advantages will be evident from the following examples, offered for illustrative purposes only. The examples are not intended to limit the scope of the present invention in any way.

### Example 1: Using Mutations of NTCP Known in Prior Art Fails to Make HepG2 Cells Susceptible for HBV

The inventors surprisingly found that it is not sufficient to “humanize” positions 157-165 of porcine NTCP as indicated in the prior art. It has been shown in Old World monkeys that a mutation of the amino acids 157-165 (G157K, R158G, I160V, L161I, P165L) of the macaque NTCP (mcNTCP) renders those monkeys susceptible for an infection with HBV. However, neither of these approaches was successful with porcine NTCP. HepG2 cells do not express sufficient amounts of NTCP to allow an infection with HBV (see, e.g. Iwamoto et al. 2014). Therefore, these human cells are a valuable tool to analyze the effect of different NTCP muteins in a human cell culture system.

HepG2 cells were transfected with plasmids coding for different variants of NTCP: human (hNTCP), porcine (pNTCP) or chimeric (pNTCP w/ hAA). After transfection, the cells were incubated for three days with 2% DMSO before they were infected with HBV at an MOI of 300. The supernatant was collected at days 4, 7 and 10 after infection and analyzed for the viral protein HBeAg by ELISA.

The results are shown in FIG. 5A. HepG2 transfected with hNTCP are susceptible to HBV while those transfected with pNTCP are not. Chimeric NTCP renders HepG2 cells susceptible to HBV if at least nucleotide sequence positions 1-572 of the porcine NTCP are human. Interestingly, a chimeric NTCP, in which amino acid sequence positions 157, 158, 164 and 166 of pNTCP are humanized, cannot render HepG2 cells susceptible to HBV, i.e. the mutations suggested in prior art are not sufficient in case of the porcine NTCP. The inventors surprisingly found that also amino acid sequence position 167 has to be “humanized”.

FIG. 5B is a graphical overview of the NTCP muteins shown in FIG. 5A. Here, the different NTCP chimeras and mutated pNTCP are shown. It is obvious that amino acid sequence positions 157-167 have to be human to arrive at phNTCP that renders the cell susceptible for HBV.

### Example 2: Making Primary Porcine Hepatocytes Susceptible for an Infection with HBV

### Material and Methods

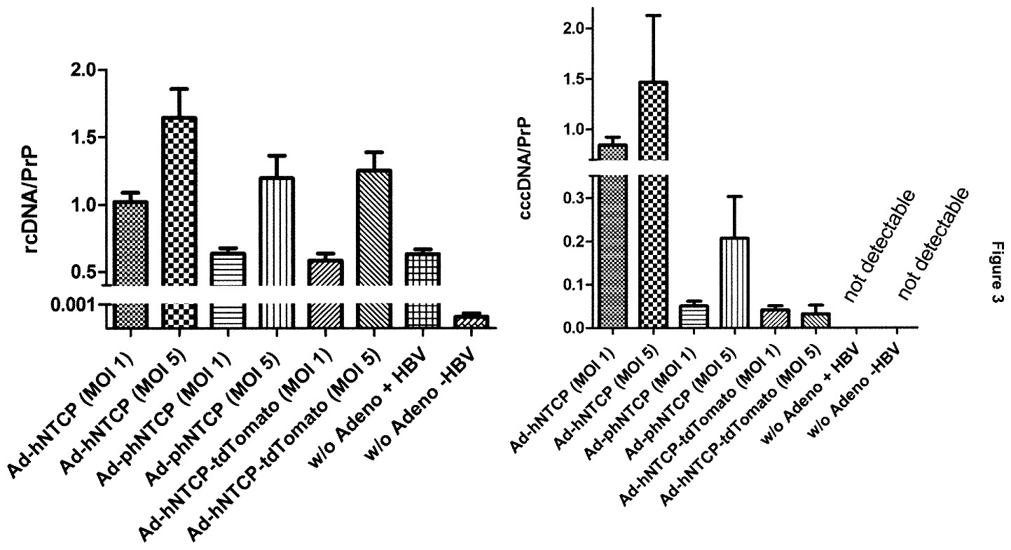

Primary porcine hepatocytes (PPH) were isolated from a pig liver less than 3 h after slaughter and kept in culture: For infection experiments, the cells were seeded in a 24 well plate and for Southern Blot experiments into 6 well plates. The next day, the cells were transduced with adenovirus at an MOI of 1 or 5 (see FIG. 2) and cultured in DMEM-F12 comprising 2 DMSO. The sequences of the adenoviruses are shown in SEQ ID NO: 6 for Ad-hNTCP, SEQ ID NO: 7 for Ad-phNTCP and SEQ ID NO: 8 for Ad-hNTCP-tdTomato.

On day 4 after isolation (day 3 after transduction), the cells were infected with HBV (genotype D, serotype ayw) in DMEM-F12 with 4% PEG6000 with an MOI of 1000.

On days 3 and 5 after HBV infection, samples were taken for HBeAg measurements. Medium was changed on day 3 after HBV infection. The 6 well plate for the Southern Blot analysis was frozen at −80° C. on day 4 after HBV infection and used for a Hirt extraction with subsequent Southern Blot. To detect protein-free forms of HBV DNA including cccDNA, a modified Hirt extraction procedure was used (Yan et al. 2012). Intracellular capsid-associated DNA was prepared as described (Ko et al. 2014). Viral DNA forms were separated on an agarose gel, transferred onto a nylon membrane, and hybridized with a digoxigenin-labeled HBV-specific probe (Ko et al. 2014). DNA signal was detected by DIG Luminescent Detection Kit (Roche).

On day 5 after infection, the 24 well plates were lysed, frozen and then DNA was isolated and analyzed for rcDNA or cccDNA using qPCR. In detail, total cellular DNA was extracted using NucleoSpin Tissue kit (Macherey Nagel). For selective cccDNA PCR, isolated DNA was treated with 5 units of T5 exonuclease (NEB, Frankfurt, Germany) for 30 min in 10 μl reaction volume (Xia 2017b) followed by heat-inactivation at 95° C. for 5 min and 4-fold dilution with distilled water. Two different primer sets were used to detect total intracellular HBV DNA (HBV1844F: 5′-GTTGCCCGTTTGTCCTCTAATTC-3′ (SEQ ID NO: 9) and HBV1745R: 5′-GGAGGGATACATAG (SEQ ID NO: 10).

phNTCP Mutein Renders Cells Susceptible for an Infection with HBV

The concentration of viral protein HBeAg in the supernatant of PPH cell cultures was determined by ELISA on day 3 and day 5 after infection with HBV. The amount of HBeAg in the supernatant is an indicator for the infection efficiency of HBV. As can be seen from FIG. 2, PPH transduced with human NTCP (Ad-hNTCP) are susceptible for an infection with HBV while untransduced PPH (w/o Adeno) are not susceptible for HBV. In addition, the porcine NTCP mutein of the present invention (Ad-phNTCP) renders also the PPH susceptible for an infection with HBV.

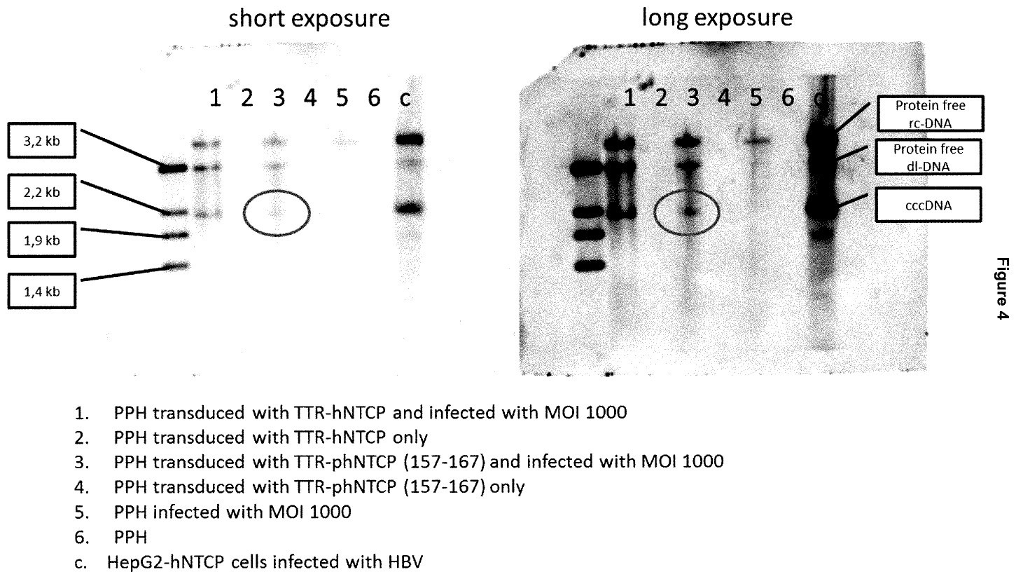

A similar picture can be observed on the level of viral DNA expression in the PPH. Here, cells were analyzed at day 5 after infection (see FIG. 3). A higher MOI leads to higher infection efficiency. In addition, cells transduced with hNTCP and the phNTCP mutein show a drastic increase of rcDNA (relaxed circular DNA) and cccDNA (covalently closed circular DNA) of HBV, while again, untransduced cells (w/o Adeno+HBV) or uninfected cells (w/o Adeno—HBV) do not express the viral DNA.

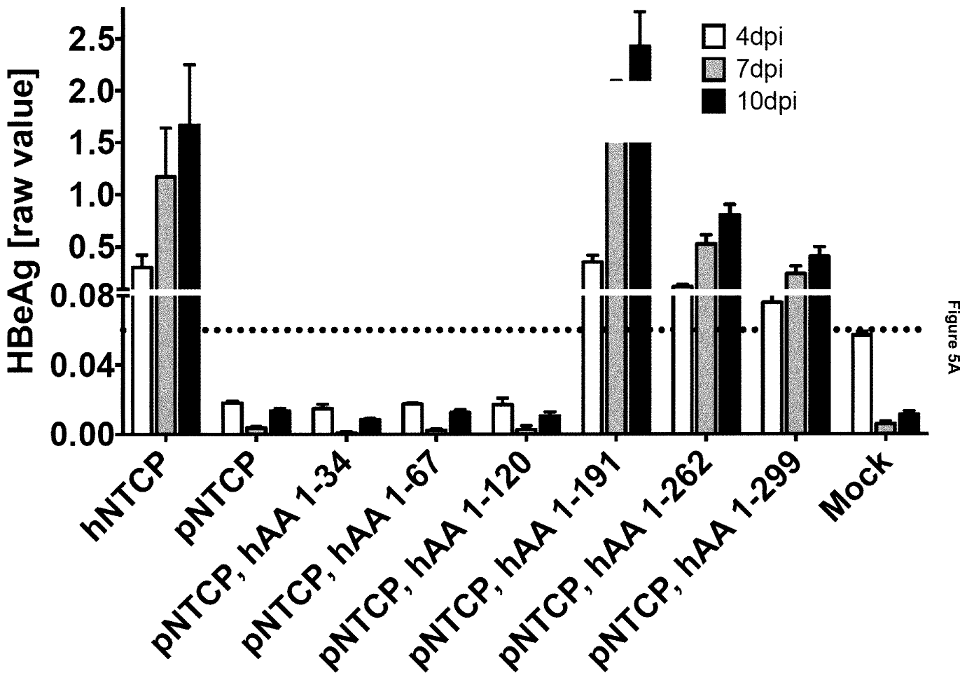

The results are confirmed when the amount of HBV cccDNA is analyzed by Southern Blot analysis. Here, the PPH were analyzed on day 4 after infection with HBV. As can be seen in FIG. 4, only PPH transduced with hNTCP (lane 1) or phNTCP (lane 3) and infected with HBV show HBV cccDNA in the Southern Blot.

In sum, it is apparent that a human NTCP is needed for a successful infection of PPH. Also the newly found humanized porcine NTCP (phNTCP) renders the PPH susceptible for an infection with HBV. Thus, the porcine NTCP mutein of the present invention is capable of rendering cells susceptible to HBV.

### Example 3: CRISPR-Mediated Mutation of Porcine Cells

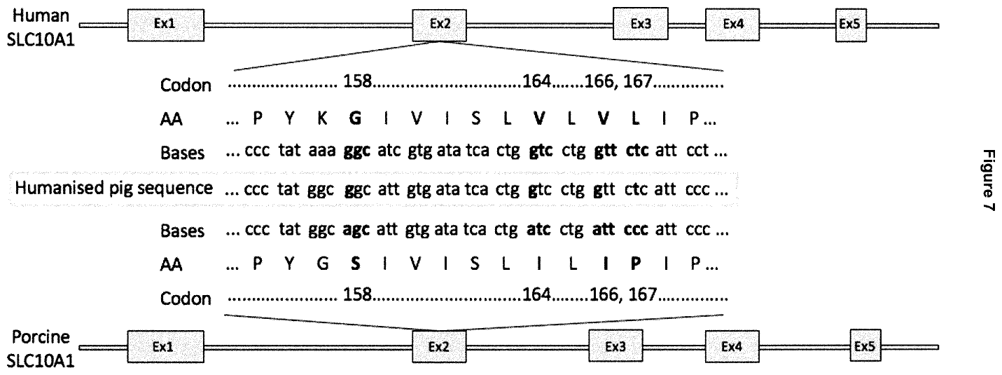

In addition to the overexpression of the NTCP muteins of the present invention as shown in Example 2, the inventors also applied a CRISPR/Cas9-mediated mutation of sequence positions 157, 158, 164, 166 and 167 of the endogenous porcine NTCP in primary porcine cells. FIG. 7 shows the strategy underlying this approach.

Here, porcine kidney fibroblasts were transfected with a modified PX330 plasmid, carrying an additional Puromycin selection cassette, porcine NTCP guide RNA sequence and Cas9 nuclease. Several guide RNA sequences (18 bp long) were tested for their efficiency to cause Insertion/Deletion (Indel) mutations.

NTCP G1
(SEQ ID NO: 20)
TCCAGGGGCATCTATGAT
NTCP G2
(SEQ ID NO: 21)
TCTATGATGGGACCCTGA
NTCP G3
(SEQ ID NO: 22)
GAAGGACAAGGTGCCCTA
NTCP G4
(SEQ ID NO: 23)
CTCCTATACCTTTACTCC
NTCP G5
(SEQ ID NO: 24)
ATCATCCTCAACACTAAA

All tested guide RNA sequences showed high Indel frequencies. Guide 3 (NTCP G3) showed the highest Indel frequency and is located closest to the porcine endogenous sequence that has to be humanized. Moreover, the PAM sequence of guide 3 consists of the last base of aa 156 (T) and the first two bases of aa 157 (GG) which had to be humanized. In case of humanization, the PAM sequence gets changed from TGG to TAA. TAA is no more recognized as PAM sequence and Cas9 does not cut the humanized sequence again. This strongly increases the efficiency of humanization. For these reasons, guide 3 was chosen for further experiments.

Porcine kidney fibroblasts were then transfected with the PX330-Puro-G3 plasmid (carrying guide 3) and a ssDNA repair oligonucleotide after 24 h of serum starvation.

SsDNA oligos were produced by using a plasmid DNA template and the Takara Guide-it Long ssDNA Production system. The DNA template consisted of 2900 bp of porcine NTCP with humanization of bases 157, 158, 164 and 166 (SEQ ID NO: 35). The left homology arm was about 1556 bp, the right homology arm about 1314 bp.

Using this DNA template for ssDNA synthesis, several ssDNA templates were prepared using the following primer combinations:

| | | Amplicon |
|---|---|---|
| | | length |
| Left primer | Right primer | in bp |
| aagcccttgtcagttgcatca | tcctccactgtataggtgaaaccaa | 2803 |
| (SEQ ID NO: 25) | (SEQ ID NO: 26) | |
| ctgggctttccacatgcttc | gtgctgggaggacatgatgc | 1563 |
| (SEQ ID NO: 27) | (SEQ ID NO: 28) | |
| tgcttcactttgcactctcgtg | gctatgtggaagcccaaggc | 2146 |
| (SEQ ID NO: 29) | (SEQ ID NO: 30) | |
| agggaggcccaaggagaaag | ggtgaggttagtgggggcaa | 991 |
| (SEQ ID NO: 31) | (SEQ ID NO: 32) | |
| tggagaaatagcacctacagacttgc | taaggcatgtcatttgggttttt | 443 |
| (SEQ ID NO: 33) | (SEQ ID NO: 34) | |

All ssDNA templates were tested for the efficiency of humanization. The longer templates (2803, 1563 and 2146 bp) showed low efficiencies, whereas very high efficiencies of almost 100% could be obtained by using the 991 bp and 443 bp templates.

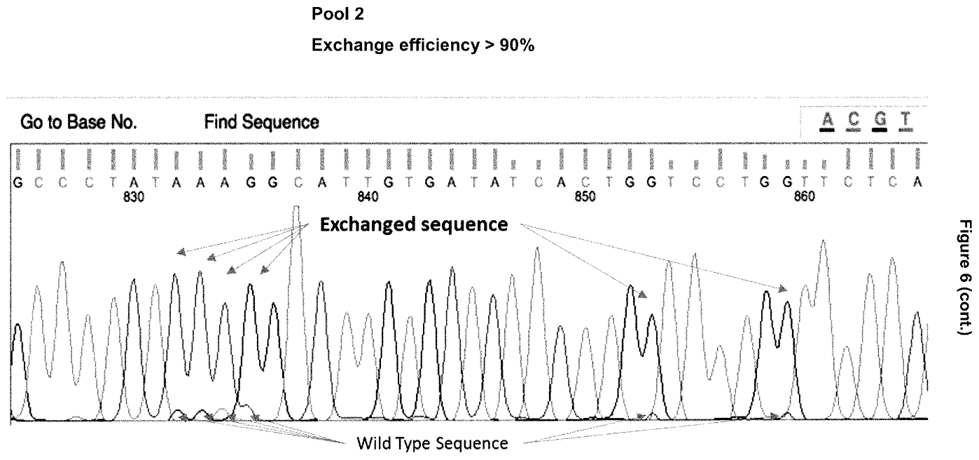

As shown in FIG. 6, the rate of successful substitutions is in the range of 70% (cell pool 1) to more than 90% (cell pool 2). Thus, the inventors were able to mutate the specific sequence positions in primary porcine cells. For generating the respective ssDNA template sequence the primer pair of SEQ ID NO: 31 and SEQ ID NO: 32 has been used, leading to the results shown in FIG. 6.

Following the mutation with CRISPR/Cas9, clones of cell pool 1 and cell pool 2 will be used for nuclear transfer to generate transgenic pigs. Alternatively, a micro injection of CRISPR/Cas9, the guideRNA and the repair template is possible.

### REFERENCES

M. Dandri, J. Petersen, Animal models of HBV infection. Best Practice & Research Clinical Gastroenterology 31, 273-279 (2017).
M. Iwamoto, K. Watashi, S. Tsukuda, H H Aly, M. Fukasawa, A. Fujimoto, R. Suzuki, H. Aizaki, T. Ito, O. Koiwai, H. Kusuhara, T. Wakita, Biochem Biophys Res Comm, 443:808-813 (2014).
Ko C, Shin Y C, Park W J, Kim S, Kim J, Ryu W S. Residues Arg703, Asp777, and Arg781 of the RNase H Domain of Hepatitis B Virus Polymerase Are Critical for Viral DNA Synthesis. J Virol, 88:154-163 (2014).
F. A. Lempp et al., Sodium taurocholate cotransporting polypeptide is the limiting host factor of hepatitis B virus infection in macaque and pig hepatocytes. Hepatology 66, 703-716 (2017).
S. Müller et al, Characterisation of the hepatitis B virus cross-species transmission pattern via Na⁺/taurocholate co-transporting polypeptides from 11 New World and Old World primate species. PLoS ONE 13(6), e0199200 (2018).
K. Watashi, S. Urban, W. Li, T. Wakita, NTCP and beyond: opening the door to unveil hepatitis B virus entry. Int J Mol Sci 15, 2892-2905 (2014).
W. He et al., Modification of Three Amino Acids in Sodium Taurocholate Cotransporting Polypeptide Renders Mice Susceptible to Infection with Hepatitis D Virus In Vivo. J Virol 90, 8866-8874 (2016).
X. Li, J. Zhao, Q. Yuan, N. Xia, Detection of HBV Covalently Closed Circular DNA. Viruses 9, (2017).
W. S. Mason, Animal models and the molecular biology of hepadnavirus infection. Cold Spring Harb Perspect Med 5, (2015).
H. Yan et al., Sodium taurocholate cotransporting polypeptide is a functional receptor for human hepatitis B and D virus. eLife 1, e00049 (2012).
H. Yan et al., Molecular determinants of hepatitis B and D virus entry restriction in mouse sodium taurocholate cotransporting polypeptide. J Virol 87, 7977-7991 (2013).
Y. Xia, U. Protzer, Control of Hepatitis B Virus by Cytokines. Viruses 9, (2017a).
Xia Y, Stadler D, Ko C, Protzer U. Analyses of HBV cccDNA Quantification and Modification. Methods Mol Biol, 1540:59-72 (2017b).

1. A porcine sodium taurocholate cotransporter polypeptide (NTCP) mutein, wherein the mutein comprises glycine at sequence position 158, valine at sequence position 164, valine at sequence position 166 and leucine at sequence position 167 relative to the sequence positions of wild type porcine NTCP depicted as SEQ ID NO: 2.

2. The NTCP mutein of claim 1, further comprising an amino acid residue selected from the group consisting of lysine, arginine, glycine and valine at sequence position 157.

3. The NTCP mutein of claim 1, wherein the mutein comprises the sequence KGIVISLVLVL depicted as SEQ ID NO: 1 at sequence positions 157-167 relative to the sequence positions of wild type porcine NTCP depicted as SEQ ID NO: 2.

4. The mutein of claim 1, wherein the mutein is capable of rendering a cell genetically modified with the mutein being susceptible to an infection with hepatitis B virus (HBV) and/or hepatitis D virus (HDV).

5. The mutein of claim 1 having at least 85%, at least 90%, at least 95%, at least 98% or at least 99% sequence identity with SEQ ID NO: 3 or 12.

6. A nucleic acid encoding the mutein of claim 1.

7. (canceled)

8. A host cell comprising at least one of the mutein of claim 1.

9-16. (canceled)

17. The mutein of claim 1, wherein the cell is porcine.

18. The host cell of claim 8, wherein the host cell is mammalian.

19. The host cell of claim 8, wherein the host cell is porcine.

20. The host cell of claim 8, wherein the host cell is a hepatocyte.

21. The NTCP mutein of claim 1, wherein the mutein comprises the sequence GIVISLVLVL depicted as SEQ ID NO: 11 at sequence positions 158-167 relative to the sequence positions of wild type porcine NTCP depicted as SEQ ID NO: 2.

22. A host cell comprising the nucleic acid of claim 6.
